SEQUENCE LISTING (1 sequence)

SEQ ID NO: 1 - protein, 8 aa, Artificial Sequence
  Description of Artificial Sequence Synthetic peptide
VATVSLPR

(19) United States
(12) Patent Application Publication
(10) Pub. No.: US 2007/0009960 A1
(43) Pub. Date: Jan. 11, 2007

(54) Characterising polypeptides

(75) Inventors: Jurgen Schafer, Lauterbach (DE); Christian Hamon, Frankfurt am Main (DE); Josef Schwarz, Frankfurt am Main (DE); Christopher Pearce, Cobham (GB)

Correspondence Address: BUCHANAN, INGERSOLL & ROONEY PC, POST OFFICE BOX 1404, ALEXANDRIA, VA, 22313-1404, US

(73) Assignee: Electrophoretices Limited, Cobham (GB)

(21) Appl. No.: 10/565,563

(22) PCT Filed: Jul. 22, 2004
(86) PCT No.: PCT/GB04/03139

§ 371 Date: Aug. 4, 2006

(30) Foreign Application Priority Data
Jul. 22, 2003 (GB) 0317123.8

(51) Int. Cl. G01N 33/53; C12Q 1/37; G01N 33/00

(52) U.S. Cl. 435/7.1; 435/23; 436/86; 530/409; 435/7.5

(57) ABSTRACT

Provided is a method for characterising an analyte by matrix assisted laser desorption ionisation (MALDI) mass spectrometry, which method comprises: (a) labelling the analyte with a light-absorbing label that absorbs light at a pre-determined frequency, to form a labelled analyte; (b) embedding the labelled analyte in a matrix formed from at least one compound that absorbs light, to form an embedded labelled analyte; (c) desorbing the embedded labelled analyte by exposing it to light having the pre-determined frequency, to form a desorbed analyte; and (d) detecting the desorbed analyte by mass spectrometry to characterise the analyte; wherein the light absorbing label comprises a fluorophore moiety, and wherein prior to detecting by mass spectrometry, the analyte is selected for detection on the basis of its fluorophore moiety.

### FIELD OF THE INVENTION

This invention relates to methods of labelling analyte molecules, particularly non-volatile biomolecules with multi-mode markers that enhance the sensitivity with which their associated analyte biomolecule is detectable by Matrix Assisted Laser Desorption Ionisation (MALDI) Mass Spectrometry, whilst also being detectable by non-mass spectrometric means. Specifically this invention relates to markers that combine a fluorescent detection capability with the ability of certain dyes to improve the sensitivity of detection by MALDI mass spectrometry of analytes labelled with these dyes.

### BACKGROUND OF THE INVENTION

Conventional techniques for determining the expression of proteins in biological samples depend on protein identification. The goal of protein expression profiling is to identify as many proteins in a sample as possible and, preferably, to determine the quantity of the protein in the sample. A typical method of profiling a population of proteins is by two-dimensional electrophoresis (2-DE) (R. A. Van Bogelen., E. R. Olson, “Application of two-dimensional protein gels in biotechnology.”, Biotechnol Annu Rev, 1:69-103, 1995). In this method a protein sample extracted from a biological sample is separated by two independent electrophoretic procedures. This first separation usually separates proteins on the basis of their iso-electric point using a gel-filled capillary or gel strip along which a pH gradient exists. Proteins migrate electrophoretically along the gradient until the pH is such that the protein has no net charge, referred to as the iso-electric point, from which the protein can migrate no further. After all of the proteins in the sample have reached their iso-electric point, the proteins are separated further using a second electrophoretic procedure. To perform the second procedure, the entire iso-electric focussing gel strip is then laid against one edge of a rectangular gel. The separated proteins in the strip are then electrophoretically separated in the second gel on the basis of their size. The proteins are thus resolved into a 2-dimensional array of spots in a rectangular slab of acrylamide. However, after separating the proteins in a sample from each other, there remains the problem of detecting and then identifying the proteins. The currently favoured approach to identify proteins is to analyse the protein in specific spots on the gel by peptide mass fingerprinting using MALDI-TOF mass spectrometry (Jungblut P, Thiede B. “Protein identification from 2-DE gels MALDI mass spectrometry.” Mass Spectrom Rev. 16:145-162, 1997), which has a restricted throughput. 2-DE technology is therefore limited by the detection capabilities of the peptide mass fingerprinting methods used in the identification of proteins in gel spots. One way to increase the number of useful protein identification from analyses carried out using current 2-D gel techniques is to compare gels from disease and control samples to identify differences and only identify the proteins present in spots that correspond to differential expression. An improved 2-D gel technology, in which two or more samples are covalently labelled with different fluorophores and then separated on the same gel has been developed (Unlu, M.; Morgan, M. E.; Minden, J. S. Electrophoresis 1997, 18, 2071-2077). This technology, referred to as DIGE allows paired samples to be compared quantitatively in the same analysis, which is highly advantageous as it avoids the reproducibility problems that plague comparisons of samples run on different gels. The fluorescently labelled proteins can be detected without additional staining steps, which are typically required to visualise proteins on gels. Avoiding staining is advantageous as many stains interfere with subsequent analysis by mass spectrometry. The DIGE process however, does not enhance the sensitivity of the MALDI TOF analysis typically used for peptide mass fingerprinting.

A typical peptide mass fingerprinting protocol involves determining the mass of the unidentified protein followed by digestion of the protein (in gel or in solution) with enzymes, such as trypsin. Trypsin cleaves polypeptides selectively at arginine and lysine residues, leaving either arginine or lysine at the C-termini of the product peptides. The positions of lysine and arginine in the sequence of a polypeptide determine where the polypeptide is cut giving rise to a characteristic series of peptides. The pattern of peptides can be easily detected by MALDI-TOF mass spectrometry. This mass spectrometric technique has a large mass range, can readily ionise large biomolecules, will preferentially produce singly charged ions and competition for ionisation with this technique is not severe, although competition can be problematic. This means that there is generally one peak in the mass spectrum for each peptide, the mass-to-charge ratio for each peak has essentially the same value as the mass of the peptide, with an added proton to ionise the peptide, and most (and sometimes all) the peptides from the tryptic digest of an unidentified protein can be analysed simultaneously. In effect the mass spectrum is a ‘bar-code’ in which the lines in the spectrum represent the masses of the characteristic cleavage peptides of the protein. For any given protein, there may be some peptides, which have the same mass as a peptide from another protein but it is very unlikely that two different proteins will give rise to the same series of peptides having the same series of masses. This means that the pattern of masses of the tryptic digest of a protein is a fairly unique identifier of that protein and is called a Peptide Mass Fingerprint (PMF). The relative uniqueness of PMFs means that databases of calculated (or theoretical) PMFs, determined from known protein sequences or sequences that have been predicted from genomic DNA or expressed sequence tags (ESTs), can be used to identify proteins in biological samples (Pappin D J C, Höjrup P and Bleasby A J, Current Biology 3: 327-332, “Rapid identification of proteins by peptide-mass fingerprinting.” 1993; Mann M, Hojrup P, Roepstorff P, Biol. Mass Spectrom 22(6): 338-345, “Use of mass spectrometric molecular weight information to identify proteins in sequence databases.” 1993; Yates J R 3rd, Speicher S, Griffin P R, Hunkapiller T, Anal Biochem 214(2): 397-408, “Peptide mass maps: a highly informative approach to protein identification.” 1993). The PMF for an unidentified protein can be compared with all of the PMFs in a database to find the best match, thereby identifying the protein. Searches of this kind can be constrained by determining the mass of the protein prior to digestion. In this way the pattern of masses of an unidentified polypeptide can be related to its sequence, which in turn can help to determine the role of a protein in a particular sample.

There are, however, many technical difficulties involved in determining the PMF for a protein. A typical protein will give rise to twenty to thirty peptides after cleavage with trypsin, but not all of these peptides will appear in the mass spectrum. The precise reasons for this are not fully understood. One factor that is believed to cause incomplete spectra is competition for protonation during the ionisation process, resulting in preferential ionisation of arginine containing peptides (Krause E. & Wenschuh H. & Jungblut P. R., Anal Chem. 71(19): 4160-4165, “The dominance of arginine-containing peptides in MALDI-derived tryptic mass fingerprints of proteins.” 1999). In addition, there are surface effects that result from the process of preparing MALDI targets. The targets are prepared by dissolving the peptide digest in a solution of the matrix material. Small droplets of the peptide/matrix solution are dropped onto a metal target and left to dry. Differences in solubility of peptides will mean that some peptides will preferentially crystallise near the top surface of the matrix where they will be desorbed more readily.

Sensitivity is also a problem with conventional protocols for identifying proteins from their PMF. To be an effective tool, it should be possible to determine a PMF for as small a sample of protein as possible to improve the sensitivity of the analysis of protein samples.

Some attempts have been made to improve the ionisation of peptides that do not contain arginine by chemical derivatisation of peptides. Conversion of lysine to homo-arginine is one approach that has met with some success (V. Bonetto et al., Journal of Protein Chemistry 16(5): 371-374, “C-terminal Sequence Determination of Modified Peptides by MALDI MS”, 1997; Francesco L. Brancia, Stephen G. Oliver and Simon J. Gaskell, Rapid Commun. in Mass Spec., 14, 2070-2073, “Improved matrix-assisted laser desorption/ionisation mass spectrometric analysis of tryptic hydrolysates of proteins following guanidination of lysine-containing peptides.” 2000; Brancia et al., Electrophoresis 22: 552-559, “A combination of chemical derivatisation and improved bioinformatics tools optimises protein identification for proteomics”, 2001). The conversion of lysine to homo-arginine introduces guanidino functionalities into all of the peptides from a tryptic digest, with the exception of C-terminal peptides, greatly improving the representation of lysine containing peptides in the MALDI-TOF mass spectra.

The derivatisation of peptides to introduce guanidino-groups is a method of improving the proton affinity of the derivatised peptide. This approach to improve sensitivity has been moderately successful in enhancing the sensitivity of detection in techniques that depend on protonation to achieve ionisation such as electrospray ionisation (ESI) and MALDI analysis. These techniques are most effective if the analyte does not already possess a functionality with a high proton affinity, e.g. oligosaccharides (Okamoto et al., Anal Chem. 69(15): 2919-2926, “High-sensitivity detection and post-source decay of 2-aminopyridine-derivatized oligosaccharides with matrix-assisted laser desorption/ionisation mass spectrometry.” 1997). However, analytes that already contain functionalities that are readily protonated, such as trypsin digested peptides, do not benefit significantly from such reagents and other methods to improve the sensitivity of these sorts of analyte are needed.

Various other reagents for derivatising peptides have also been developed. Reagents that introduce quaternary ammonium functionalities and quaternary phosphonium functionalities have been developed for positive ion mass spectrometry. Halogenated compounds, particularly halogenated aromatic compounds are well known electrophores, i.e. they pick up thermal electrons very easily. A variety of derivatisation reagents based on fluorinated aromatic compounds (Bian N. et al., Rapid Commun Mass Spectrom 11(16): 1781-1784, “Detection via laser desorption and mass spectrometry of multiplex electrophore-labelled albumin.” 1997) have been developed for electron capture detection, which is a highly sensitive ionisation and detection process that can be used with negative ion mass spectrometry (Abdel-Baky S. & Giese R. W., Anal Chem. 63(24):2986-2989, “Gas chromatography/electron capture negative-ion mass spectrometry at the zeptomole level.” 1991). A fluorinated aromatic group could also be used as a sensitivity-enhancing group. Aromatic sulphonic acids have also been used for improving sensitivity in negative ion mass spectrometry.

Each type of derivatisation reagent that has been disclosed in the prior art has different benefits and limitations, which depend on the method of ionisation used and on the methods of mass analysis used (for a review see Roth et al., Mass Spectrometry Reviews 17:255-274, “Charge derivatisation of peptides for analysis by mass spectrometry”, 1998). The mechanism by which sensitivity is enhanced may also be different for each type of group. Some derivatisation methods increase basicity and thus promote protonation and charge localisation, which improves sensitivity in surface desorption techniques like Matrix Assisted Laser Desorption Ionisation (MALDI) and Fast Atom Bombardment (FAB). So far, reagents that introduce charge-carrying functionalities or functionalities with high proton affinities have been developed for MALDI mass spectrometry but no reagents that improve the desorption of the analyte by increasing its ability to absorb light and to mix with the matrix have been reported.

Negative ion mass spectrometry is sometimes more sensitive because there is less background noise. A tag that can enhance both negative ion mode detection and positive ion mode detection would have significant advantages. A tag for uniformly improving sensitivity of all associated analytes has yet to be found for all mass spectrometry techniques and it is unlikely that a universal reagent will be found. However, for specific mass spectrometric techniques it should be possible to design reagents that take advantage of features of a particular technique to promote detection sensitivity.

In this invention, derivatisation reagents and methods for their use have been developed to allow proteins to be both labelled and detected on gels, whilst in addition enhancing the sensitivity of detection by MALDI mass spectrometry.

WO 98/31830 describes arrays of cleavable labels that are detectable by mass spectrometry which identify the sequence of a covalently linked nucleic acid probe. These mass labels have a number of advantages over other methods of analysing nucleic acids. At present commercially favoured systems are based on fluorescent labelling of DNA. Fluorescent labelling schemes permit the labelling of a relatively small number of molecules simultaneously; typically 4 labels can be used simultaneously and possibly up to eight. However the costs of the detection apparatus and the difficulties of analysing the resultant signals limit the number of labels that can be used simultaneously in a fluorescence detection scheme. An advantage of using mass labels is the possibility of generating large numbers of labels, which have discrete peaks in a mass spectrum allowing similar numbers of distinct molecular species to be labelled simultaneously. Fluorescent dyes are expensive to synthesize whereas mass labels can comprise relatively simple polymers permitting combinatorial synthesis of large numbers of labels at low cost. This application describes the use of mass-modified MALDI matrix molecules for the labelling of biomolecules. Tags comprising MALDI matrix agents such as cinnamic and sinnapinic acid can be attached to biomolecules through a photo-cleavable linker allowing cleavage and desorption of tags within a laser desorption ionisation mass spectrometer without requiring additional matrix.

WO 99/60007 discloses mass tags comprising trityl functionalities for the labelling of nucleic acids and oligonucleotides. These tags can be cleaved from their associated oligonucleotides by photolysis in a MALDI-TOF mass spectrometer prior to desorption. The cleavage product is charged which is advantageous as it improves the sensitivity of the detection of the tags. This method also does not require additional matrix.

The prior art therefore discloses methods and reagents for cleavable tags for use in MALDI mass spectrometry that may be desorbed without additional matrix. The present invention is distinguished by the fact that the tags are not cleaved from the analyte and that the tags may be used in the presence of free matrix material.

### SUMMARY OF THE INVENTION

It is an aim of the present invention to solve the problems associated with the above prior art. In particular, it is an object of this invention to provide methods and labels that can be used to produce improved peptide mass fingerprints and which will improve the sensitivity of detection of other labelled non-volatile macromolecules particularly other biomolecules such as proteins and nucleic acids. The methods improve sensitivity and can increase the number of peptides (e.g. small peptides) which are not detectable in conventional MALDI experiments that are detected from a protein. In addition, through the use of appropriate tags, it is possible with this invention to analyse multiple samples simultaneously and it is also possible to determine the ratios of corresponding peptides in the different samples, and also the quantity of individual peptides in one or more samples. With appropriate labelling procedures, it is also possible facilitate the conditioning of polypeptide sample for detection by mass spectrometry.

It is a further aim of this invention to provide compounds, which have desirable features as mass labels, and methods for the use of those compounds to provide improved mass spectra of associated analytes.

Accordingly, the present invention provides a method for characterising an analyte by matrix assisted laser desorption ionisation (MALDI) mass spectrometry, which method comprises:
(a) labelling the analyte with a light-absorbing label that absorbs light at a pre-determined frequency, to form a labelled analyte;
(b) embedding the labelled analyte in a matrix formed from at least one compound that absorbs light, to form an embedded labelled analyte;
(c) desorbing the embedded labelled analyte by exposing it to light having the pre-determined frequency, to form a desorbed analyte; and
(d) detecting the desorbed analyte by mass spectrometry to characterise the analyte;
   wherein the light absorbing label comprises a fluorophore moiety, and wherein prior to detecting by mass spectrometry, the analyte is selected for detection on the basis of its fluorophore moiety.

One example of the selection referred to above would include analysing the fluorophores on a number of species identified beforehand in a gel or HPLC run, and selecting a specific protein or proteins from the gel or HPLC run for examination by mass spectrometry on the basis of the specific fluorophores attached to those proteins.

Thus, the present invention provides a method of enhancing MALDI sensitivity using sensitiser mass tags (SMTs) that allows differential quantitation of analytes (e.g. proteins) from different samples in the same experiment. Quantitation may be achieved in a number of ways. These include comparison of the fluorophore fluorescence intensity on a gel or in an HPLC run; comparison of peak height/area in the MALDI spectrum; and inclusion of a mass reporter group onto a label on the analyte and measuring the quantity on the basis of the reporter group identified in a tandem mass spectrometric analysis.

In the present invention, there is a further advantage to the use of the SMTs when the method is combined with liquid chromatography (LC) rather than 2-D electrophoresis (2DE). This is particularly so for highly basic and/or small (e.g. 10 Da or less) analytes. The present method allows users with relatively simple MS and LC equipment to obtain reliable quantitation without the need for highly expensive electrospray mass spectrometry.

In the context of the present invention, MALDI is intended to encompass and type of desorption ionisation technique, or laser desorption ionisation technique, of which MALDI is merely the most common. Thus the term includes MALDI itself, MALDI-TOF, and SELDI (described below).

In a particularly preferred embodiment, the above method comprises, prior to detecting by mass spectrometry, selecting the analyte for detection on the basis of the identity, and/or quantity of its fluorophore moiety. Thus, for example, when a single sample is to be analysed, the proteins in the sample may be labelled as described above and first separated on a gel (e.g. on the basis of size and/or isoelectric point). Normally, it is not desirable to try to identify every protein revealed on the gel, but to be selective. For example, only highly expressed proteins may be of interest. In that case, the quantity of fluorophore may be used to select the proteins to extract from the gel and identify by digestion followed by mass spectrometry. Alternatively, if there are several samples being analysed together, fluorophores may be used to distinguish proteins from different samples and selection may be made on this basis. Of course, the quantity and identity may form the basis of selection if desired.

Preferably, the fluorophore moiety comprises a dye moiety. The dye moiety may be selected from xanthene dye moieties (such as a fluorescein moiety or a rhodamine moiety) and a cyanine dye moieties. More preferably the fluorophore moiety comprises a propyl-Cy3-N-hydroxysuccinimide ester, a methyl-Cy5-hydroxysuccinimide ester, or a Cy2 N-hydroxysuccinimide ester.

Generally the desorbed analyte is directly detected by mass spectrometry. Alternatively, the desorbed analyte may be indirectly detected by mass spectrometry. In this embodiment, the analyte is additionally labelled with a mass label relatable to the analyte, and the mass label is cleaved from the desorbed analyte and detected by mass spectrometry to characterise the analyte.

Typically, the light to which the embedded labelled analyte is exposed is laser light. It is preferred that the compound forming the matrix absorbs light at the same frequency as the light-absorbing label. In some embodiments, the matrix and the light-absorbing label may be formed from the same compound.

Generally, the matrix is a solid matrix or a liquid matrix. Preferably, when the matrix is a liquid matrix, it comprises nitrobenzyl alcohol. In other embodiments it is preferred that the matrix comprises a compound selected from 3-hydroxypicolinic acid, 2,5-dihydroxybenzoic acid and 4-hydroxy-alpha-cyanocinnamic acid.

The pH of the matrix is not especially limited, and the matrix may comprise an acid matrix or a basic matrix.

Typically, the light-absorbing label is formed from a dye. Preferably, the dye is a non-fluorescent dye. The dye may be selected from any suitable compound, including 4-dimethylaminoazobenzine-4′-sulphonyl chloride (DABSYL chloride), 3-hydroxypicolinic acid, 2,5-dihydroxybenzoic acid and 4-hydroxy-alpha-cyanocinnamic acid.

The nature of the analyte is not especially limited. Preferably, the analyte comprises one or more compounds selected from a protein, a polypeptide, a peptide, a peptide fragment and an amino acid.

The present invention also provides a method for characterising a polypeptide, which method comprises the steps of:
(a) optionally reducing cysteine disulphide bridges in the polypeptide to form free thiols, and capping the free thiols;
(b) cleaving the polypeptide with a sequence specific cleavage reagent to form peptide fragments;
(c) optionally deactivating the cleavage reagent;
(d) capping one or more amino groups that are present with a lysine reactive agent;
(e) analysing peptide fragments according to a method as defined above to form a mass fingerprint for the polypeptide; and
(f) determining the identity of the polypeptide from the mass fingerprint.

Further provided is a method for characterising a population of polypeptides, which method comprises the steps of:
(a) optionally reducing cysteine disulphide bridges in one or more polypeptides to form free thiols, and capping the free thiols;
(b) separating one or more polypeptides from the population;
(c) cleaving one or more polypeptides with a sequence specific cleavage reagent to form peptide fragments;
(d) optionally deactivating the cleavage reagent;
(e) capping one or more ε-amino groups that are present with a lysine reactive agent;
(f) analysing peptide fragments according to a method as defined above to form a mass fingerprint for one or more polypeptides; and
(g) determining the identity of one or more polypeptides from the mass fingerprint.

The invention also provides a method for comparing a plurality of samples, each sample comprising one or more polypeptides, which method comprises the steps of:
(a) optionally reducing cysteine disulphide bridges and capping the free thiols in one or more polypeptides from the samples;
(b) separating one or more polypeptides from each of the samples;
(c) cleaving the polypeptides with a sequence specific cleavage reagent to form peptide fragments;
(d) optionally deactivating the cleavage reagent;
(e) capping one or more s-amino groups that are present with a lysine reactive agent;
(f) analysing peptide fragments according to a method as defined above to form a mass fingerprint for one or more polypeptides from the samples; and
(g) determining the identity of one or more polypeptides in the samples from one or more mass fingerprints.

In the above methods, it is preferred that the lysine-reactive agent is a labelled lysine-reactive agent.

The above methods may be adapted to a plurality of samples if desired. One such method according to this invention, for comparing a plurality of samples, each sample comprising one or more polypeptides, comprises the steps of:
(a) optionally reducing cysteine disulphide bridges and capping the free thiols in one or more polypeptides from the samples;
(b) capping one or more ε-amino groups that are present in each sample with a labelled lysine reactive agent;
(c) pooling the samples;
(d) separating one or more polypeptides from the pooled samples;
(e) cleaving the polypeptides with a sequence specific cleavage reagent to form peptide fragments;
(f) optionally deactivating the cleavage reagent;
(g) analysing peptide fragments according to a method as defined above to form a mass fingerprint for one or more polypeptides from the samples; and
(h) determining the identity of one or more polypeptides in the samples from one or more mass fingerprints.
   wherein the same label is employed for polypeptides or peptides from the same sample, and different labels are employed for polypeptides or peptides from different samples, such that the sample from which a polypeptide or peptide originates can be determined from its label.

Preferably the sequence specific cleavage agent cleaves the one or more polypeptides on the C-terminal side of a lysine residue. The specific cleavage reagent typically comprises Lys-C or Trypsin. Generally, the peptide fragments having capped ε-amino groups are removed by affinity capture. In this embodiment the lysine reactive agent may comprise biotin.

In some preferred embodiments, the lysine reactive agent comprises a hindered Michael reagent. Typically, the hindered Michael agent comprises a compound having the following structure: 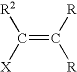
wherein X is an electron withdrawing group that is capable of stabilising a negative charge; the R groups independently comprise a hydrogen, a halogen, an alkyl, an aryl, or an aromatic group with the proviso that at least one of the R groups comprises a sterically hindering group; and the group R² comprises a hydrogen, a halogen, a hydrocarbon group, an electron withdrawing group and/or a linker capable of attachment to an affinity capture functionality or a solid phase support.

Further provided by the invention is a labelled analyte compound, which compound has either of the following structures:

F-D-L-A
D-F-L-A

wherein F comprises a fluorophore, D comprises a light absorbing label, L comprises a linker and A comprises an analyte. Typically, the fluorophore F is attached to D via a further linker. If desired, a mass marker M may be situated between D or F and L (F-D-M-L-A, or D-F-M-L-A), especially if the label is itself to be analysed by mass spectrometry.

Also provided is a compound for labelling an analyte, which compound has either of the following structures:

F-D-L-R
D-F-L-R

wherein F comprises a fluorophore, D comprises a light absorbing label, L comprises a linker, and R comprises a reactive functionality for attaching the compound to an analyte. Typically, the fluorophore F is attached to D via a further linker. If desired, a mass marker M may be situated between D or F and L (F-D-M-L-R, or D-F-M-L-R), especially if the label is itself to be analysed by mass spectrometry.

In the case of all of the above types of compound, it is generally preferred that if M is present, each label has the same M, or at least an M having the same mass, so that the spectrum is not complicated by multiple peaks derived from multiple masses for M.

Generally, D comprises a non-fluorescent dye, as already described above. Thus, D may comprise, for example a cinnamic acid derivative, a nicotinic acid derivative, a picolinic acid derivative, a hydroxybenzoic acid derivative, a methoxybenzoic acid derivative or a sinapinic acid derivative. Preferably, the non-fluorescent dye comprises a compound selected from 4-dimethylaminoazobenzine4′-sulphonyl chloride (DABSYL chloride), 3-hydroxypicolinic acid, 2,5-dihydroxybenzoic acid and 4-hydroxy-alpha-cyanocinnamic acid.

The nature of M is not especially limited. Preferably, M is selected from a compound formed from an aryl ether, and an oligomer formed from 2 or more aryl ether units.

The linker is also not especially limited. Preferably, the linker, and/or the further linker, comprises a group selected from —CR₂—CH₂—SO₂—, —N(CR₂—CH₂—SO₂—)₂, —NH—CR₂—CH₂—SO₂—, —CO—NH—, —CO—O—, —NH—CO—NH—, —NH—CS—NH—, —CH₂—NH—, —SO₂—NH—, —NH—CH₂—CH₂— and —OP(═O)(O)O—.

Typically, the analyte, A, is selected from a protein, a polypeptide, a peptide, a peptide fragment and an amino acid.

The fluorophore is preferably a moiety as already defined above.

Typically, R comprises an ester group, an acid anhydride group, an acid halide group such as an acid chloride, an N-hydroxysuccinamide group, a pentafluorophenyl ester group, a maleimide group, an alkenyl sulphone group, or an iodoacetamide group.

The invention further provides a kit for characterising an analyte by matrix assisted laser desorption ionisation (MALDI) mass spectrometry, which kit comprises:
(a) one or more light absorbing labels having a reactive functionality for attaching the labels to an analyte, as defined above; and
(b) a compound for forming a matrix, which compound absorbs light at the same frequency as the light-absorbing label.

The invention will now be described in more detail, by way of example only, with reference to more specific embodiments. In one aspect of this invention there is provided a tag compound that comprises a MALDI Dye linked to a fluorophore, both of which are linked to a reactive functionality.

In one embodiment of this aspect of the invention the label molecules have one of the following structures:
MALDI Dye—Linker-Fluorophore-Linker-Reactive Functionality
Fluorophore—Linker-MALDI Dye-Linker-Reactive Functionality

Where the MALDI dye is preferably non-fluorescent and preferably dissipates absorbed radiation thermally.

In a further embodiment of this aspect of the invention, an array of two or more tag compounds is provided where each different tag compound is differentiated by the fluorophore, which has a different emission frequency from the other tag compounds. In preferred embodiments, the array of tag compounds comprises tags with the same mass.

In a further aspect of this invention there is provided a method of analysing one or more samples of analyte molecule comprising the steps of:
1. Labelling each sample of analyte molecules with a different tag compound of the first aspect of the invention that comprises a MALDI Dye and a fluorophore, where each different tag compound is differentiated by the Fluorophore, which has a different emission frequency from the other tag compounds.
2. Separating the labelled analytes
3. Detecting the labelled analytes by measurement of fluorescence.
4. Isolating the labelled analytes.
5. Optionally, cleaving the labelled analyte molecule
6. Embedding the labelled analyte molecule in a matrix comprising a dye that absorbs light at the frequency of the laser to be used in the desorption step.
7. Desorbing the labelled analyte by application of laser light of the predetermined frequency so as to effect sublimation of the matrix and thus also the labelled analyte.
8. Detecting ions formed during the desorption step by mass spectrometry.

In a still further aspect of this invention these is provided a kit comprising:
1. A mass label molecule according to the first aspect of this invention
2. A compatible MALDI matrix reagent.

In the following, a more detailed description of the invention is provided by way of example only, with reference to the Figures, in which:

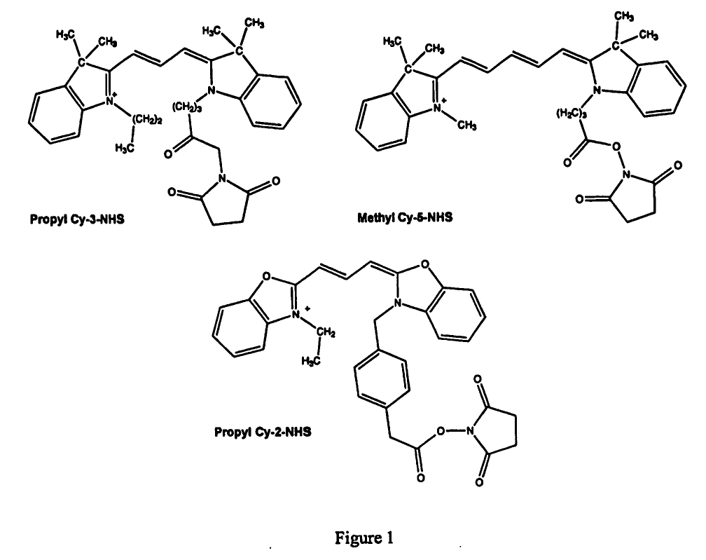

FIG. 1 shows three commercially available reactive fluorophores, propyl-Cy3-N-hydroxysuccinimide ester, methyl-Cy5-hydroxysuccinimide ester and the Cy2 N-hydroxysuccinimide ester—these fluorophores will react with amino groups in proteins—corresponding proteins in different samples labelled with these dyes will co-migrate;

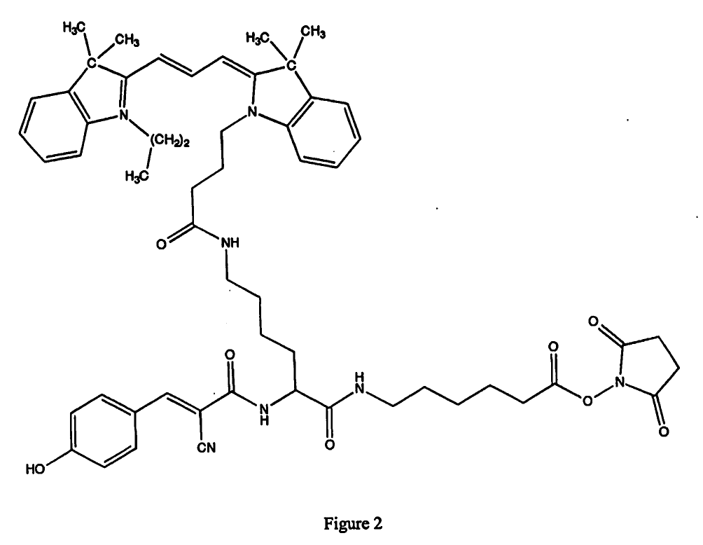

FIG. 2 shows a bimodal tag comprising the propyl-Cy3 fluorophore linked to a cinnamic acid derivative and an amine reactive N-hydroxysuccinimide ester functionality;

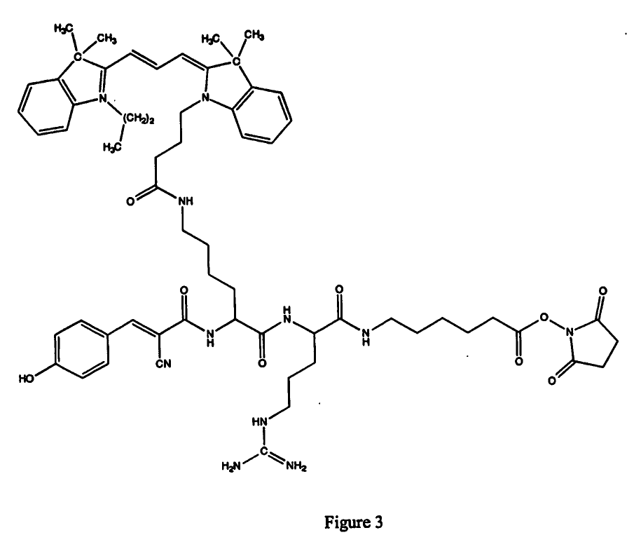

FIG. 3 shows a bimodal tag comprising the propyl-Cy3 fluorophore linked to a cinnamic acid derivative, arginine for solubilisation and an amine reactive N-hydroxysuccinimide ester functionality;

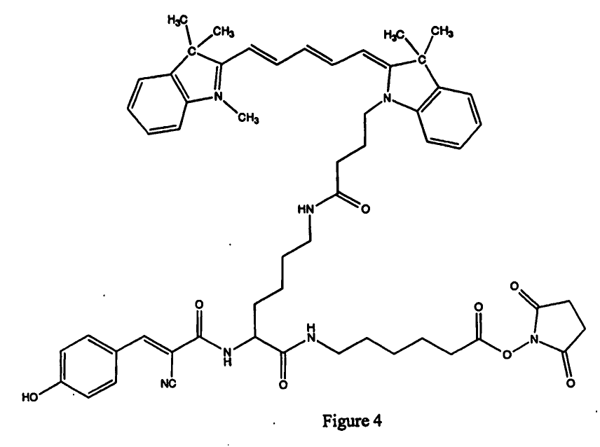

FIG. 4 shows a bimodal tag comprising the methyl-Cy5 fluorophore linked to a cinnamic acid derivative and an amine reactive N-hydroxysuccinimide ester functionality;

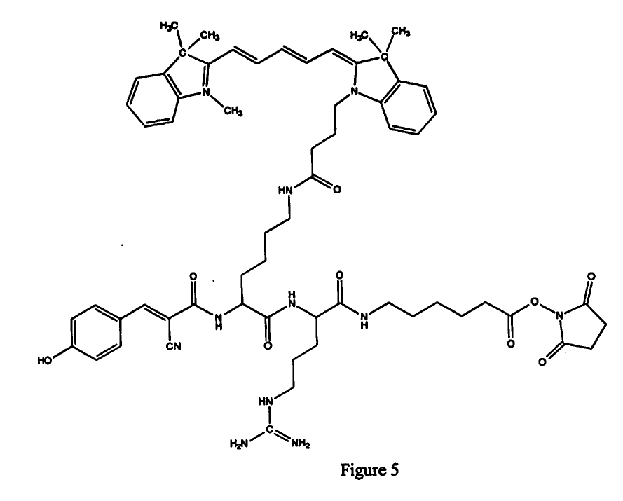

FIG. 5 shows a bimodal tag comprising the methyl-Cy5 fluorophore linked to a cinnamic acid derivative, arginine for solubilisation and an amine reactive N-hydroxysuccinimide ester functionality;

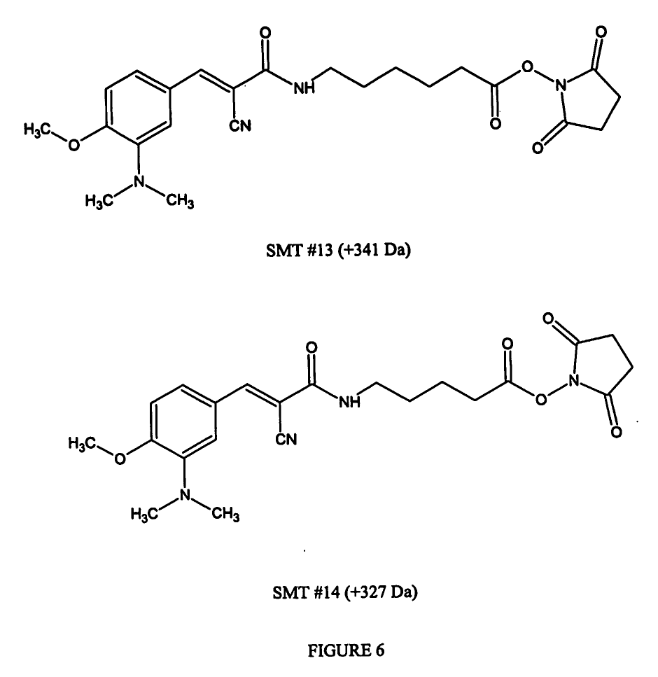

FIG. 6 shows two tags of the present invention (SMT #13 and SMT #14) which may be employed in an embodiment of the present invention capable of determining the quantities of peptide present—the SMT tags #13 and #14 differ in the length of the linker between the aromatic system and the reactive group, resulting in a mass difference of 14.0156 Da;

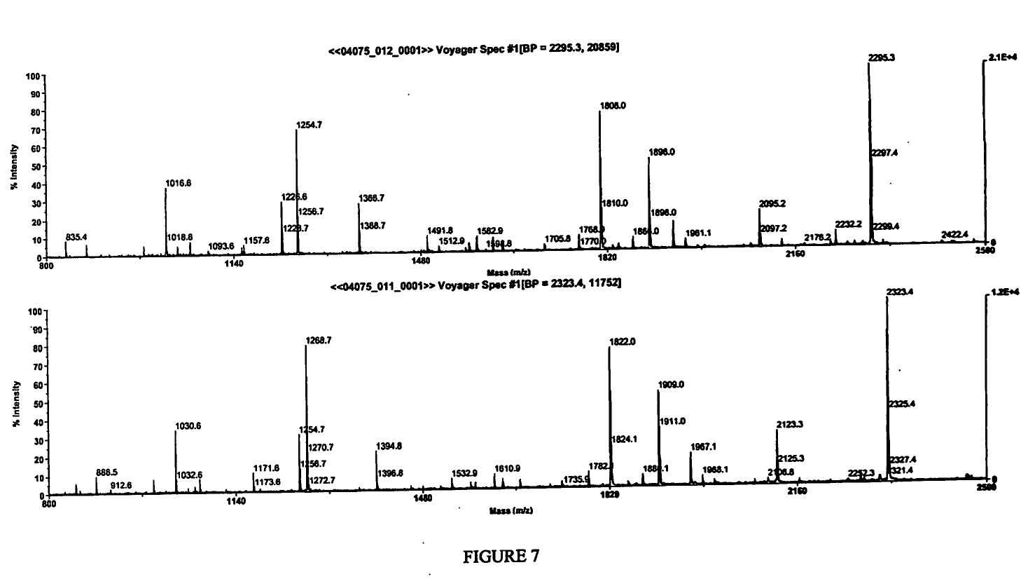

FIG. 7 shows two mass spectra of a BSA digest for comparison, the upper employing SMT #14 and the lower employing SMT #13;

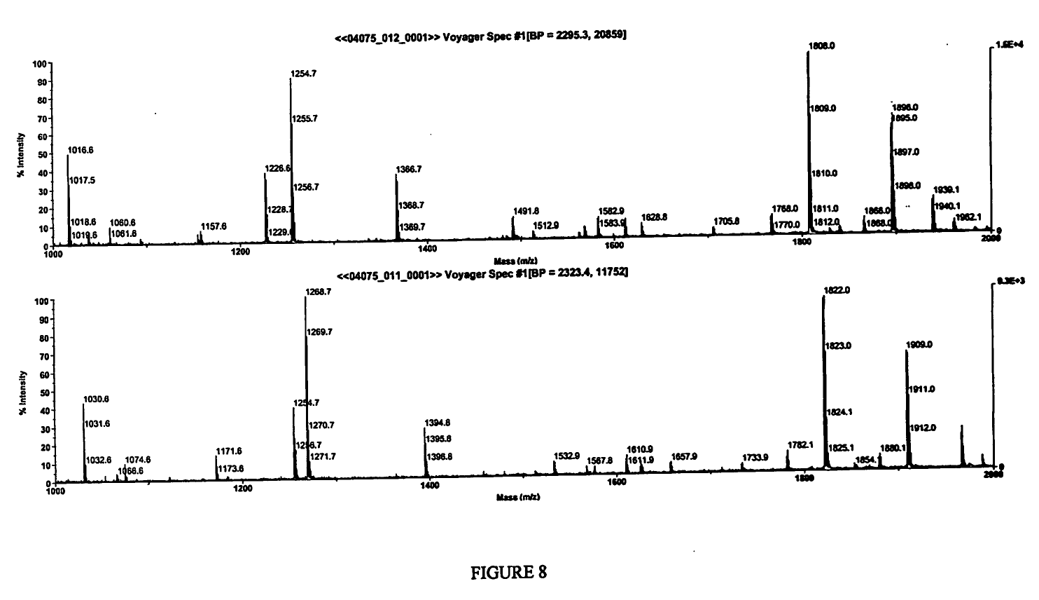

FIG. 8 shows an expanded view of the spectra in FIG. 7;

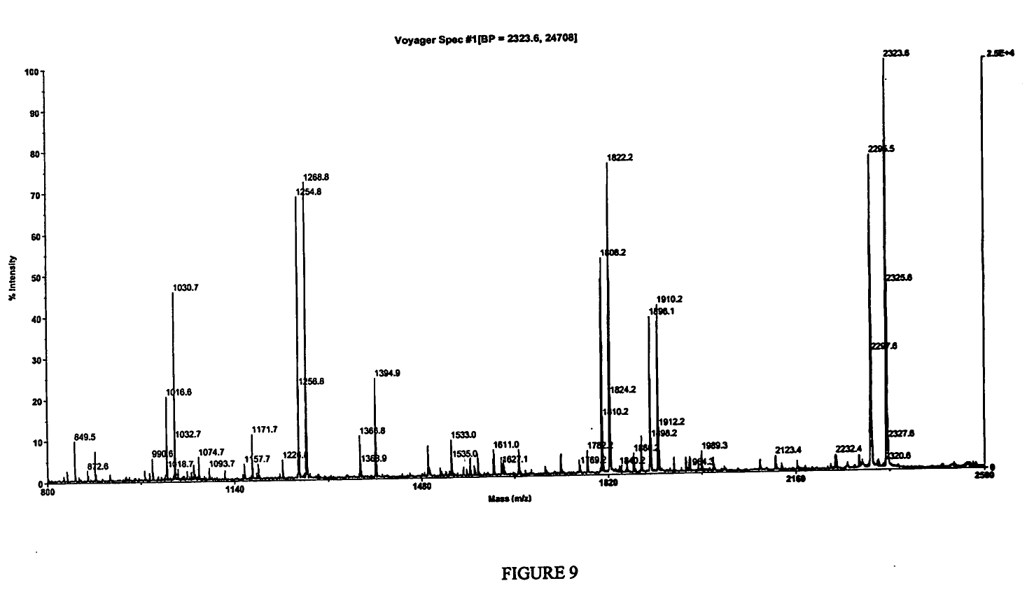

FIG. 9 shows a single mass spectrum of a 1:1 mixture of an SMT #13 labelled BSA digest and an SMT #14 labelled digest;

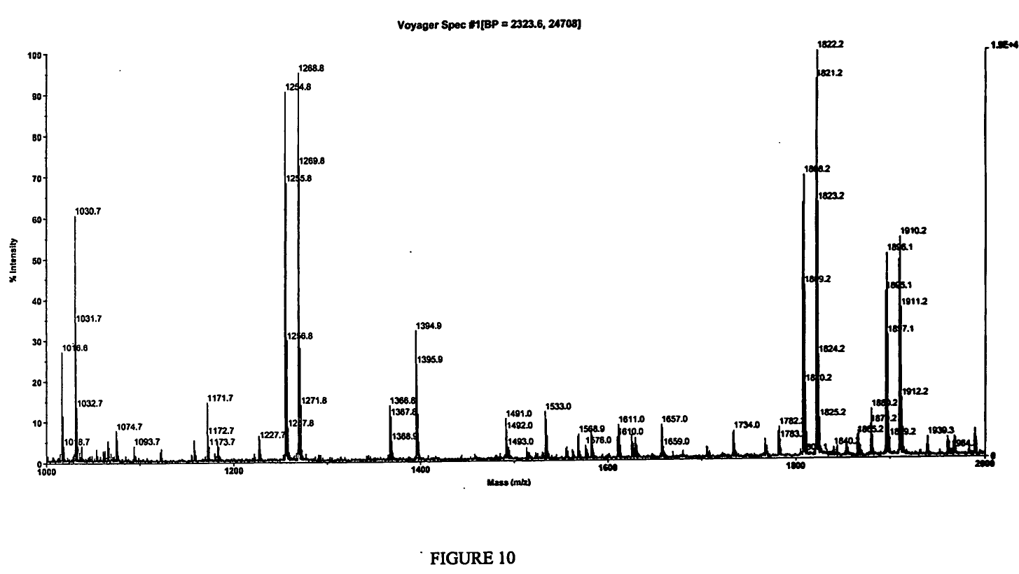

FIG. 10 shows an expanded view of the spectrum in FIG. 9;

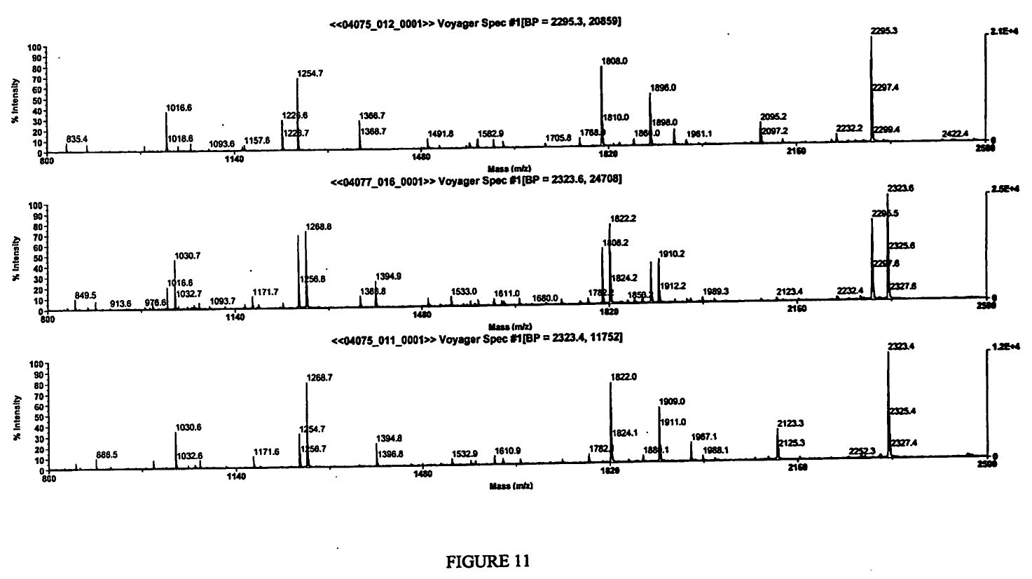

FIG. 11 shows three spectra of BSA labelled with different SMTs, the upper with SMT #13, the lower with SMT #14 and the centre a 1:1 mixture of both;

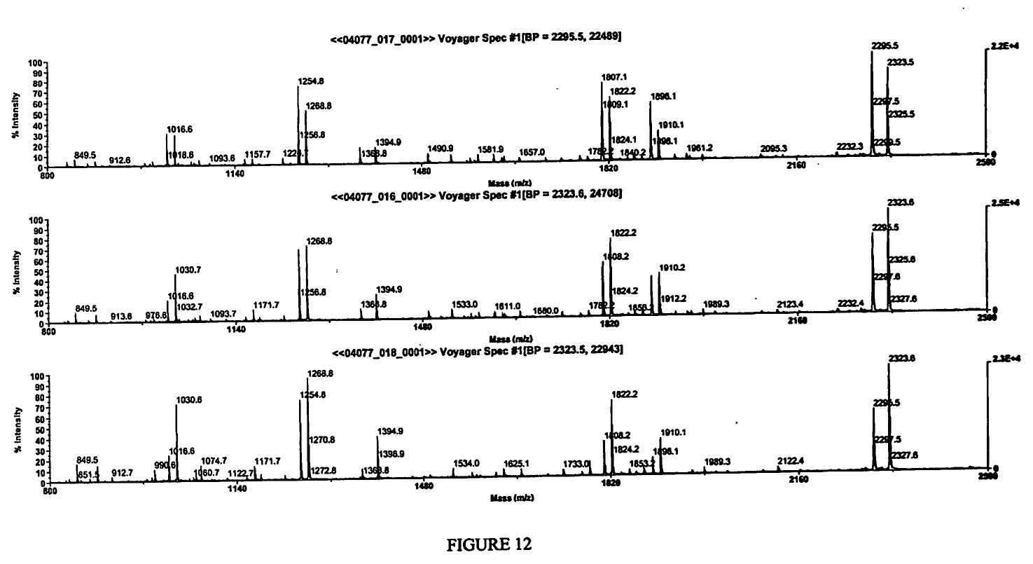

FIG. 12 shows three spectra of BSA labelled with both SMTs, in differing ratios of #13:#14, the upper with 1:2, the centre with 1:1 and the lower with 2:1;

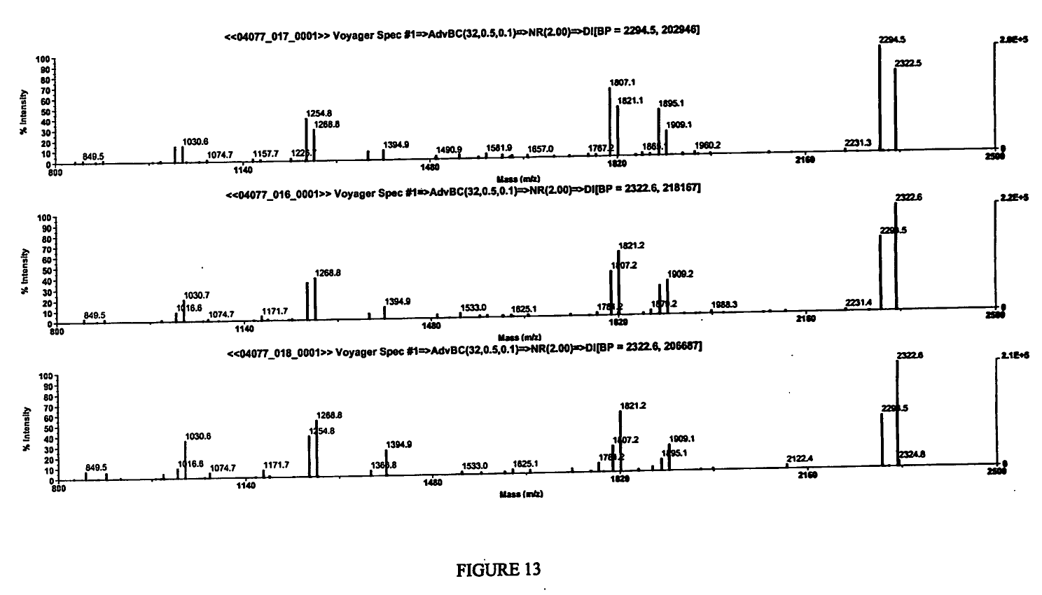

FIG. 13 shows the three spectra of FIG. 12 after de-isotoping;

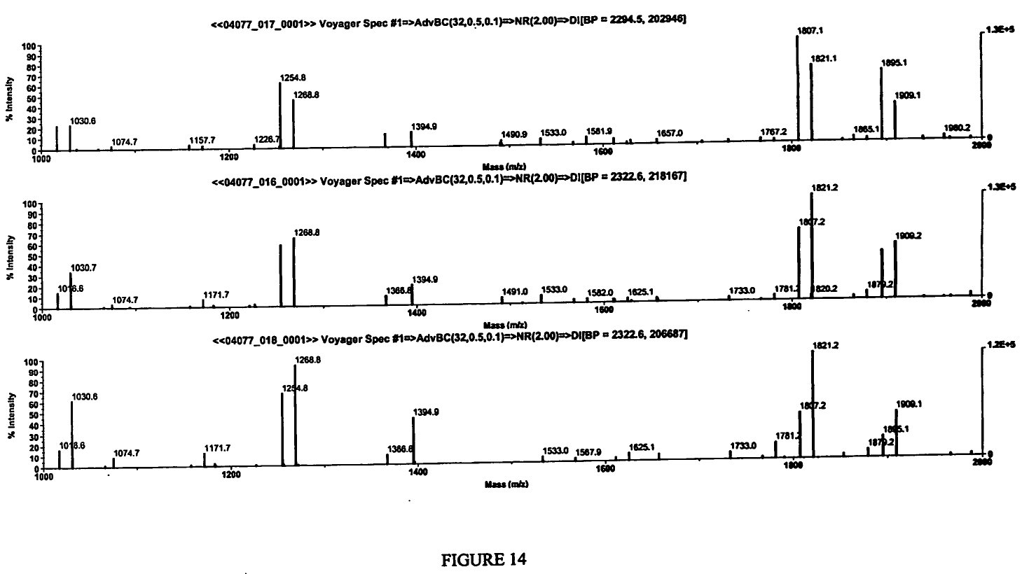

FIG. 14 shows an expanded view of the spectrum of FIG. 13;

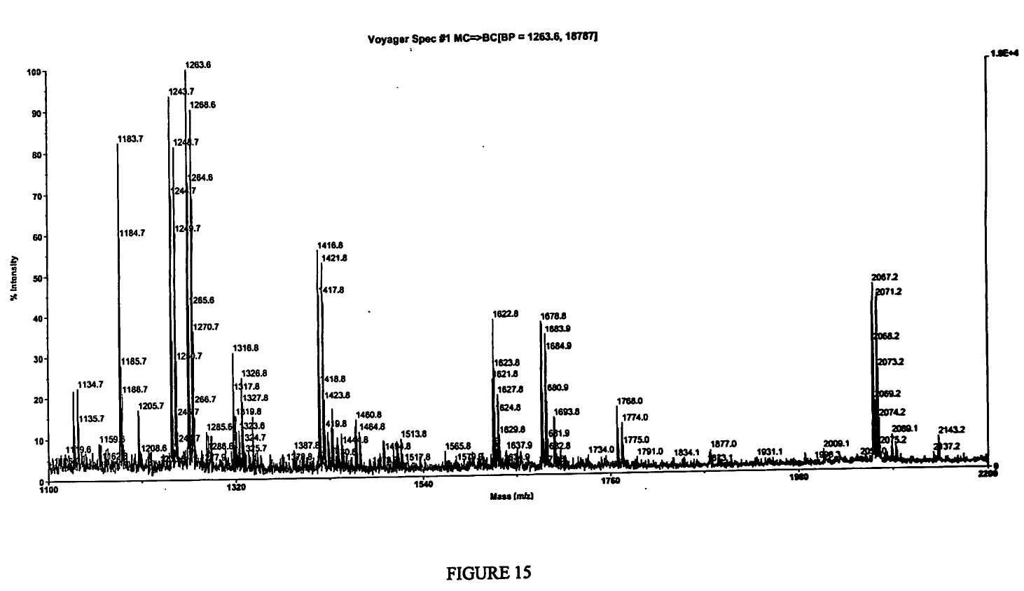

FIG. 15 shows a spectrum of the results of a BSA digest which has been labelled both with a quantitative protein sequence tag (qPST) and a tag of the present invention (SMT)—the qPST labels are differentially isotopically labelled, resulting in pairs of peaks, the higher mass of the pair from one sample, and the lower mass of the pair from the other sample;

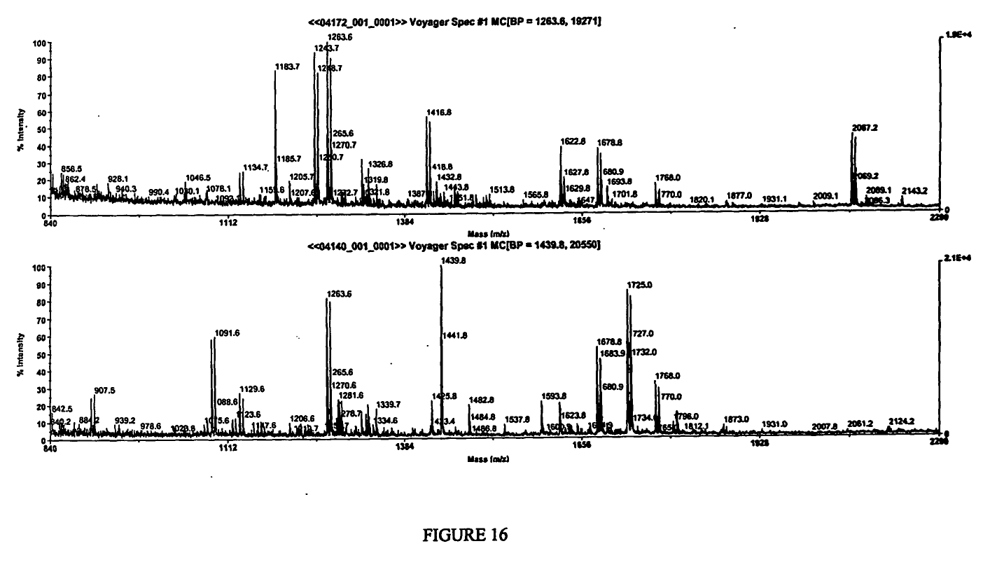

FIG. 16 shows a comparison of the same digest as in FIG. 15 with and without the sensitising tags of the present invention;

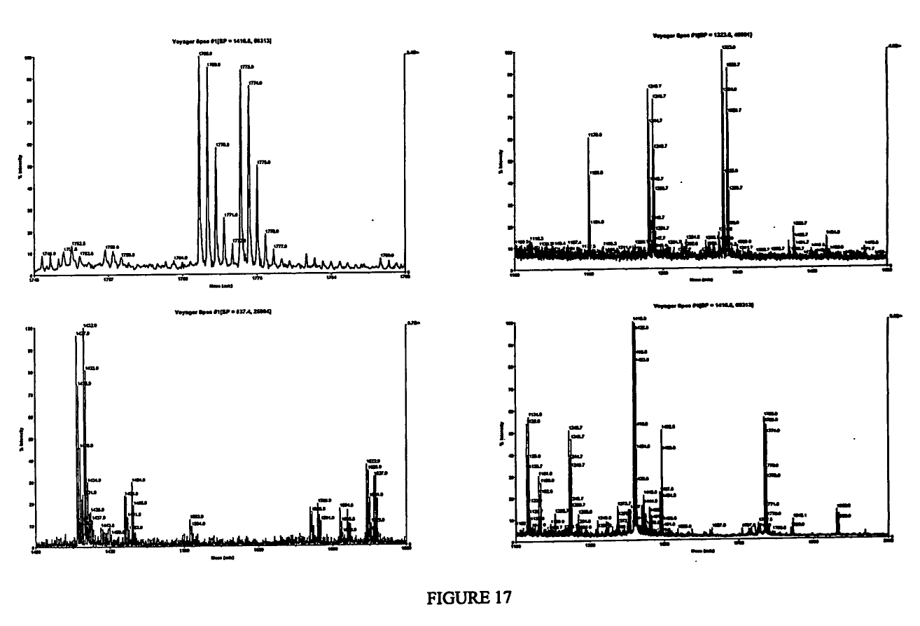

FIG. 17 shows selected ion pairs after including an HPLC separation step in the process;

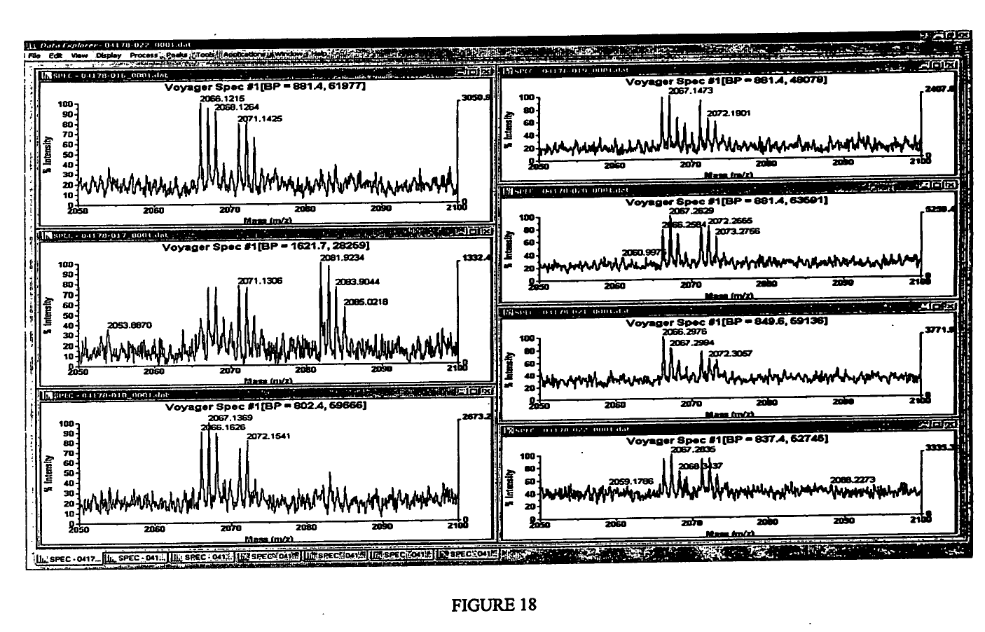

FIG. 18 shows that even a weak pair of peaks in the spectrum may give rise to useful quantitation data using a sensitizer of the present invention; and

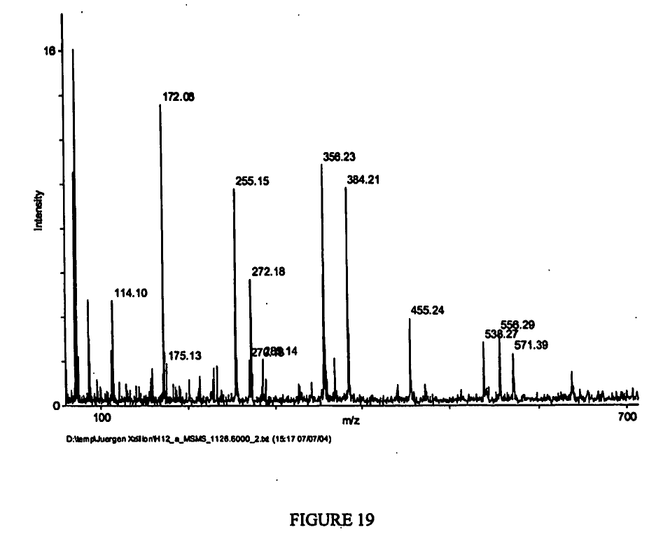

FIG. 19 shows a tandem mass spectrum of the sensitiser mass tagged (SMT) peptide VATVSLPR.

### DETAILED DESCRIPTION OF THE INVENTION

The invention will now be described in further detail.

MALDI Matrix Dyes

Various compounds have been found as matrices for MALDI analysis of large biomolecules. These compounds are generally characterised by a number of properties. The compounds generally have a strong extinction coefficient at the frequency of the laser used for desorption. The compounds are also able to isolate analyte molecules in a solid solution and the compounds are sufficiently volatile to rapidly sublime when exposed to laser shots in the MALDI mass spectrometer. The subliming dye should vaporise rapidly in a jet that entrains the embedded analyte molecules and for most purposes this should take place without fragmentation of the analyte (although fragmentation may sometimes be desirable if structural information about the analyte is sought). However, a matrix should not be too volatile as experiments can sometimes take several hours and the analyte/matrix co-crystal must remain stable under vacuum in the ion source for this period of time. The properties of volatility under laser irradiation and stability under vacuum conflict to some extent. The property of volatility to laser irradiation can be measured approximately by determining the initial velocity of analyte ions generated by the matrix. It has been observed that higher initial velocities correspond to ‘softer’ ionisation, i.e. reduced fragmentation, (Karas M. & Gluckmann M., J. Mass Spectrom. 34: 467-477, “The initial ion velocity and its dependence on matrix, analyte and preparation method in Ultraviolet Matrix-assisted Laser Desorption/lonisation”, 1999) but high initial ion velocities of some matrices also correlates to rapid sublimation under vacuum.

Different matrices have different properties in terms of their ability to assist in the desorption of embedded analytes and in the subsequent sensitivity with which the analytes are detected. It has been found empirically that certain matrices are more appropriate for the analysis of particular analytes than others. For example, 3-hydroxypicolinic acid has been found to be most effective for analysing oligonucleotides (Wu et al., Rapid Commun. Mass Spectrom. 7:142-146, “Matrix-assisted laser desorption time-of-flight mass spectrometry of oligonucleotides using 3-hydroxypicolinic acid as an ultra-violet sensitive matrix”, 1993), while 2,5-dihydroxybenzoic acid and 4-hydroxy-alpha-cyano-cinnamic acid (HCCA) are both most effective for the analysis of peptides and proteins (Strupat et al., Int. J. Mass Spectrom. Ion Proc. 111: 89-102, “2,5-dihroxybenzoic acid: a new matrix for laser desorption/ionisation mass spectrometry”, 1991; Beavis et al., Org. Mass Spectrom. 27: 156-158, “∝-cyano-4-hydroxy cinnamic acid as a matrix for matrix-assisted laser desorption mass spectrometry”, 1992). Various cinnamic acid derivatives have been found to be effective for the analysis of proteins (Beavis R. C. & Chait B. T., Rapid Commun Mass Spectrom 3(12): 432-435, “Cinnamic acid derivatives as matrices for ultraviolet laser desorption mass spectrometry of proteins.” 1989) and the choice of matrix is dependant on the nature of the analyte, for example sinnapinic acid is sometimes preferred over HCCA for large peptides and polypeptides, while HCCA is generally preferred for smaller peptides. 2,5-dihydroxybenzoic acid seems to produce less fragmentation than the cinnamic acid derivatives in some cases. The choice of matrix for a given analyte often requires some experimentation to achieve optimal results.

Most of the matrices discussed above have been acidic matrices. Basic matrices have also been developed and may be more appropriate for the analysis of acid-sensitive compounds (Fitzgerald et al., Anal Chem. 65(22): 3204-3211, “Basic matrices for the matrix-assisted laser desorption/ionisation mass spectrometry of proteins and oligonucleotides.” 1993).

Infrared MALDI (IR-MALDI) is similar in principal to ultraviolet MALDI (UV-MALDI) in that analytes must be embedded in a matrix that preferably has a strong extinction coefficient at the frequency of the laser in the desorption instrument. Appropriate matrices tend to be different compounds from those used in UV-MALDI and liquid matrices are often used. Glycerol, urea, ice and succinic acid have all been shown to be effective matrices for IR-MALDI (Talrose et al., Rapid Commun Mass Spectrom 13(21): 2191-2198, “Insight into absorption of radiation/energy transfer in infrared matrix-assisted laser desorption/ionisation: the roles of matrices, water and metal substrates.” 1999). However, some UV-MALDI matrices, such as cinnamic acid derivatives, also appear to work as IR matrices (Niu et al., J. Am. Soc. Mass Spectrom. 9:1-7, “Direct comparison of infrared and ultraviolet wavelength matrix-assisted laser desorption/ionisation mass spectrometry of proteins”, 1998).

Liquid matrices for UV-MALDI have also been explored (Ring S. & Rudich Y., Rapid Commun Mass Spectrom 14(6): 515-519, “A comparative study of a liquid and a solid matrix in matrix-assisted laser desorption/ionisation time-of-flight mass spectrometry and collision cross section measurements.” 2000; Sze et al., J Am Soc Mass Spectrom 9(2): 166-174, “Formulation of matrix solutions for use in matrix-assisted laser desorption/ionisation of biomolecules.” 1998; Karas et al. Mass Spectrom Rev 10: 335, 1991). The simplest examples of liquid matrices comprise solutions of the matrices used as solids. True liquid matrices are also known such as nitrobenzoyl alcohol. Both types of matrix have some advantages in terms of sample consistency, stability under vacuum and ease of handling however solid matrices still tend to be more sensitive. In the context of the present invention, the improvements in sensitivity may justify the use of liquid matrices. This may have advantages in the automation of sample preparation, as liquid handling robotics are widely available and the use of solutions of matrices, for solid matrix co-crystallisation, which readily clog dispensing devices can be avoided.

Reactive Tags Comprising Dyes and MALDI Matrix Dyes

In the first aspect of this invention reactive dye molecules are provided. Various dyes that are not conventionally used in MALDI mass spectrometry may be used with this invention. Some dyes that absorb strongly in UV frequencies are commercially available with reactive functionalities, e.g. 4-dimethylaminoazobenzene-4′-sulfonyl chloride (DABSYL Chloride, Sigma-Aldrich, Poole, Dorset, UK). It is anticipated by the inventors that this reagent and similar UV absorbing dyes that thermally dissipate luminal excitation should be applicable with this invention.

It is also possible to prepare reactive dyes from commercially available intermediates. A number of acidic matrices that are widely used for MALDI mass spectrometry, such as cinnamic, nicotinic and hydroxybenzoic acid derivatives, are commercially available. The acidic functionality in most of these reagents is a carboxylic acid group. This functionality may be readily converted to an active ester or acid chloride by conventional chemical methods (see for example Solomons, “Organic Chemistry”, Fifth Edition published by Wiley). Preferred active esters include N-hydroxysuccinimide (NHS) esters and pentafluorophenyl esters. It is anticipated by the inventors that a variety of dyes will function in the methods of this invention, but it is expected that preferred compounds will absorb strongly in the frequency that is used for laser desorption in the mass spectrometer. Typically Ultra-Violet (UV) radiation is used. Infared lasers are also used for MALDI applications.

Cinnamic acid derivatives are preferred dyes that are widely used in UV-MALDI TOF (Beavis R C, Chait B T, Rapid Commun Mass Spectrom 3(12):432-435, “Cinnamic acid derivatives as matrices for ultraviolet laser desorption mass spectrometry of proteins.” 1989). A reactive derivative of cinnamic acid is discussed in the examples below. It is anticipated that this reagent may be applicable to both UV- and IR-MALDI.

Reactive Functionalities

Proteins contain various nucleophilic functionalities that can be labelled using reagents that are reactive to these functionalities. Proteins typically contain thiol, amino, hydroxyl and imidazole groups. These may all be labelled with appropriate reagents if desired. In preferred embodiments of this invention, amino groups are labelled. Amino groups may be labelled with a variety of labels but acid chlorides and active esters are usually the most selective reactive functionalities. A variety of other reactive functionalities may be appropriate to prepare the reactive dyes of this invention. Table 1 below lists some reactive functionalities that may be incorporated into a dye molecule. These reactive functionalities may be reacted with nucleophilic functionalities which are found in biomolecules, particularly in peptides and polypeptides. Reaction of the reactive functionalities with the nucleophilic functionalities shown generates a covalent linkage between the two entities. This covalent linkage is shown in the third column of the table. For applications involving synthetic oligonucleotides, primary amines or thiols are often introduced during the synthesis at the termini of the molecules to permit labelling. Any of the functionalities listed below could be introduced into the compounds of this invention to permit the mass markers to be attached to a molecule of interest. A reactive functionality can be used to introduce a further linker groups with a further reactive functionality if that is desired. Table 1 is not intended to be exhaustive and the present invention is not limited to the use of only the listed functionalities.

TABLE 1

| Nucleophlic Functionality | Reactive Functionality | Resultant Linking Group |
|---|---|---|
| —SH | —SO₂—CH═CR₂ | —S—CR₂—CH₂—SO₂— |
| —NH₂ | —SO₂—CH═CR₂ | —N(CR₂—CH₂—SO₂—)₂ or |
| | | —NH—CR₂—CH₂—SO₂— |
| —NH₂—OH | 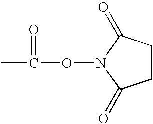 | —CO—NH——CO—O— |
| —NH₂—OH | 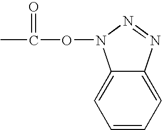 | —CO—NH——CO—O— |
| —NH₂ | —NCO | —NH—CO—NH— |
| —NH₂ | —NCS | —NH—CS—NH— |
| —NH₂ | —CHO | —CH₂—NH— |
| —NH₂ | —SO₂Cl | —SO₂—NH— |
| —NH₂ | CH₂═CH— | —NH—CH₂—CH₂— |
| —OH | —OP(NCH(CH₃)₂)₂ | —OP(═O)(O)O— |

It should be noted that in applications involving labelling oligonucleotides with the mass markers of this invention, some of the reactive functionalities above or their resultant linking groups might have to be protected prior to introduction into an oligonucleotide synthesiser. Preferably unprotected ester, thioether and thioesters, amine and amide bonds are to be avoided, as these are not usually stable in an oligonucleotide synthesiser. A wide variety of protective groups is known in the art, which can be used to protect linkages from unwanted side reactions. Alternatively, amine functionalised oligonucleotides can be prepared using standard methods known in the art and these can be labelled with amine reactive reagents such as NHS-esters.

Fluorophores

In the first aspect of this invention, a tag compound is provided that comprises a fluorophore. Numerous fluorophores are known in the art and most are applicable with this invention. Preferred dyes however include xanthene dyes (such as fluorescein and rhodamine dyes) and cyanine dyes (see for example U.S. Pat. No. 5,286,486 which is incorporated herein by reference).

In preferred embodiments of this invention, two or more samples, e.g. protein samples, are labelled with different tag reagents as defined in the first aspect of this invention and the labelled samples are analytically separated, e.g. by 2-D gel electrophoresis. The separated analytes are located and their relative quantities are determined by measurement of the fluorescence from the tags. In these embodiments, the different fluorescent tags preferably meet certain criteria

The reagents preferably have matching size and charge so that labelled analytes co-migrate during analytical separations. The fluorophores are advantageous if they have a high quantum yield and a high coefficient of absorption. The fluorophores should preferably be pH insensitive, i.e. there show no change in signal over wide pH range used during first dimension (IEF) separation. The emission frequencies of the fluorophores should preferably not overlap so that a discrete signal is obtained from each fluorophore. In addition, the fluorophores are advantageously photostable to minimize loss of signal during labelling, analytical separation and detection.

Preferred dyes for use with this invention (see FIG. 1) are disclosed in U.S. Pat. No. 6,127,134 and Mujumdar et al. (Bioconjug Chem. 4(2):105-11 “Cyanine dye labelling reagents: sulfoindocyanine succinimidyl esters.”, 1993). These documents disclose indole containing dyes with distinct emission frequencies, high coefficients of absorption and high quantum yields. The dyes have also been size and charge matched. These documents disclose active esters of these dyes for the labelling of amino groups in proteins, particularly lysine epsilon amino groups. The lysine amino acid in proteins carries an intrinsic single positive charge at neutral or acidic pH. The fluorophores disclosed in U.S. Pat. No. 6,127,134 also carry a single positive charge which, when coupled to the lysine, replaces the lysine's single positive charge with its own, ensuring that the pI of the labelled protein does not significantly alter compared to the same unlabelled protein. The active ester dyes disclosed in this application can easily be coupled to a linker to allow these fluorophores to be incorporated into the tag compounds of the present invention (see FIGS. 2 to 5).

Affinity Capture Ligands

In certain embodiments of this invention the mass markers may additionally comprise an affinity capture ligand. Affinity capture ligands are ligands, which have highly specific binding partners. These binding partners allow molecules tagged with the ligand to be selectively captured by the binding partner. Preferably a solid support is derivatised with the binding partner so that affinity ligand tagged molecules can be selectively captured onto the solid phase support. The use of an affinity capture ligand provides many advantages, in that tagged species can be selectively captured prior to analysis allowing separation of tagged and untagged material while also allowing for conditioning of the analyte for mass spectrometry. Conditioning of a sample may include removal of detergents and other contaminants that can suppress ionisation or otherwise interfere with mass spectrometry. Conditioning also includes removal of salts that may form adducts with analytes causing mass shifts in the mass spectrum. In addition, pH may be adjusted to optimise ionisation. Conditioning of tagged analytes captured onto a solid phase support is trivial as the captured material can be easily washed with an appropriate buffer comprising volatile salts such as ammonium carbonate or trifluoroacetic acid depending on the desired pH. This washing step can remove contaminants and can be used to adjust the pH appropriately.

A further advantage of the inclusion of an affinity ligand is the ability to selectively isolate certain analyte species if the tag additionally comprises a reactive functionality that will couple the affinity ligand to specific analytes. For example, Gygi et al. (Nature Biotechnology 17: 994-999, 1999) disclose the use of ‘isotope encoded affinity tags’ (ICAT) for the capture of peptides from proteins, to allow protein expression analysis. The authors report that a large proportion of proteins (>90%) in yeast have at least one cysteine residue (on average there are ˜5 cysteine residues per protein). Reduction of disulphide bonds in a protein sample and capping of free thiols with iodoacetamidylbiotin results in the labelling of all cysteine residues. The labelled proteins are then digested, with trypsin for example, and the cysteine-labelled peptides may be isolated using avidinated beads. These captured peptides can then be analysed by liquid chromatography tandem mass spectrometry (LC-MS/MS) to determine an expression profile for the protein sample. Two protein samples can be compared by labelling the cysteine residues with a different isotopically modified biotin tag. In a useful embodiment of this invention, isotopically differentiated, cysteine reactive tags of this invention comprising an affinity ligand could be employed to improve the sensitivity of the ICAT analysis method. Similarly, Schmidt and Thompson (WO 98/32876) disclose the use biotin reagents to capture C- or N-terminal peptides for protein expression profiling analysis by mass spectrometry. The sensitivity of this process would also be enhanced by tags of this invention comprising an affinity ligand.

A preferred affinity capture ligand is biotin, which can be introduced into the tags of this invention by standard methods known in the art. In particular a lysine residue may be incorporated after amino acid 2 through which an amine-reactive biotin can be linked to the peptide mass tags (see for example Geahlen R. L. et al., Anal Biochem 202(1): 68-67, “A general method for preparation of peptides biotinylated at the carboxy terminus.” 1992; Sawutz D. G. et al., Peptides 12(5): 1019-1012, “Synthesis and molecular characterization of a biotinylated analog of [Lys]bradykinin.” 1991; Natarajan S. et al., Int. J. Pept. Protein Res. 40(6): 567-567, “Site-specific biotinylation. A novel approach and its application to endothelin-1 analogs and PTH-analog.”, 1992). Iminobiotin and desthiobiotin are also applicable. A variety of avidin counter-ligands for biotin are available, which include monomeric and tetrameric avidin and streptavidin, all of which are available on a number of solid supports.

Other affinity capture ligands include digoxigenin, fluorescein, nitrophenyl moieties and a number of peptide epitopes, such as the c-myc epitope, for which selective monoclonal antibodies exist as counter-ligands. Metal ion binding ligands such as hexahistidine, which readily binds Ni²⁺ ions, are also applicable. Chromatographic resins, which present iminodiacetic acid chelated Ni²⁺ ions are commercially available, for example. These immobilised nickel columns may be used to capture tagged peptide, which comprise oligomeric histidine. As a further alternative, an affinity capture functionality may be selectively reactive with an appropriately derivitised solid phase support. Boronic acid, for example, is known to selectively react with vicinal cis-diols and chemically similar ligands, such as salicylhydroxamic acid. Reagents comprising boronic acid have been developed for protein capture onto solid supports derivitised with salicylhydroxamic acid (Stolowitz M. L. et al., Bioconjug Chem. 12(2): 229-239, “Phenylboronic Acid-Salicylhydroxamic Acid Bioconjugates. 1. A Novel Boronic Acid Complex for Protein Immobilization.” 2001; Wiley J. P. et al., Bioconjug Chem. 12(2): 240-250, “Phenylboronic Acid-Salicylhydroxamic Acid Bioconjugates. 2. Polyvalent Immobilization of Protein Ligands for Affinity Chromatography.” 2001, Prolinx, Inc, Washington State, USA). It is anticipated that it should be relatively simple to link a phenylboronic acid functionality to the tags of this invention to generate capture reagents that can be captured by selective chemical reactions. The use of this sort of chemistry would not be directly compatible with biomolecules bearing vicinal cis-diol-containing sugars, however these sorts of sugars could be blocked with phenylboronic acid or related reagents prior to reaction with boronic acid derivitised tag reagents.

Charge Derivatisation of Peptides

In some embodiments of the first aspect of this invention the tags may comprise readily ionisable groups, which can assist both in solubilisation of the tag and tagged analytes and in ionisation of the tagged analytes in the mass spectrometer. Various functionalities can be used as ionisable groups. The tertiary amino group and the guanidino group are both useful functionalities for solubilisation and ionisation (Francesco L. Branca, Stephen G. Oliver and Simon J. Gaskell, Rapid Commun. in Mass Spec., 14, 2070-2073, “Improved matrix-assisted laser desorptionl/ionisation mass spectrometric analysis of tryptic hydrolysates of proteins following guanidination of lysine-containing peptides.” 2000).

Various other methods for derivatising peptides have been also been developed. These include the use of quaternary ammonium derivatives, quaternary phosphonium derivatives and pyridyl derivatives for positive ion mass spectrometry. Each type of ionisable functionality has different benefits, which depend on the method of ionisation used and on the methods of mass analysis used. Some derivitisation reagents increase basicity and thus promote protonation and/or charge localization for positive ion mass spectrometry while other reagents readily lose protons making them appropriate for negative ion mass spectrometry, which is often more sensitive than positive ion mass spectrometry because there is less background noise. Charge derivitisation can also change the fragmentation products of derivatised peptides, when collision induced dissociation is used. In particular some derivatisation techniques simplify fragmentation patterns, which is highly advantageous, if peptides are to be analysed by techniques such as collision induced dissociation. The choice of ionising functionality will be determined by the mass spectrometric techniques that will be employed (for a review see Roth et al., Mass Spectrometry Reviews 17:255-274, “Charge derivatisation” of peptides for analysis by mass spectrometry”, 1998). For the purposes of this invention ionising functions that promote positive or negative ion formation are equally applicable.

Charged groups such as tertiary amino functionalities, guanidino functionalities and sulphonic acid functionalities provide an additional advantage. These groups can act as affinity ligands allowing tagged analytes to be purified by ion exchange. Tags comprising guanidino functions (see for example FIGS. 3 and 5) and tertiary amino functions can be captured onto a strong cation exchange resin allowing conditioning prior to mass spectrometry analysis. Similarly, tags comprising sulphonic acid functions can be captured onto anion exchange resins allowing conditioning prior to mass spectrometry analysis. Additionally, the interaction between unreacted tags and the resin, anion or cation exchange, is weaker than the interaction of tagged analytes allowing unreacted tag to be readily washed away. The tagged analytes can be eluted with an appropriate buffer comprising a suitable concentration of a volatile acid, base or salt depending on the resin. Accordingly, it is envisaged that pipette tips, spin columns and cartridges packed with a cation exchange resin or an anion exchange resin will be useful tools for the preparation of labelled samples to allow facile clean-up of the labelled peptides prior to analysis.

In addition sulphonic acid groups are advantageous for MALDI TOF analysis. Sulphonic acid derivatives of the alpha-amino functionality of peptides have been shown to enhance fragmentation efficiency in MALDI-Ion Trap analysis of peptides with improved spectra for certain classes of peptides that typically give poor MS/MS spectra in the ion trap, such as peptides containing aspartic and glutamic acid (Keough, T., Lacey M. P., et al., Rapid Commun Mass Spectrom 15(23): 2227-2239, “Atmospheric pressure matrix-assisted laser desorption/ionisation ion trap mass spectrometry of sulphonic acid derivatised tryptic peptides.”, 2001). The strongly acidic functionality facilitates protonation of the amide backbone of singly charged peptides in MALDI leading to increased fragmentation.

In specific embodiments of this invention, tags comprising guanidino groups and sulphonic acid groups have been synthesized (see Figures and the examples section). In general preferred charged groups include guanidino groups, tertiary amino groups and sulphonic acid groups.

Surface Enhanced Laser Desorption Ionisation

Surface Enhanced Laser Desorption lonisation (SELDI) is a variant of MALDI in which the usual metal targets for MALDI are derivatised (Weinberger S. R., Morris T. S., Pawlak M., Pharmacogenomics 1(4):395-416 “Recent trends in protein biochip technology.”, 2000). These surface modifications include derivatisation with anion exchangers, cation exchangers, hydrophobic surfaces or hydrophilic surfaces. When protein or peptide samples are applied to these surfaces, the sample adsorbs to the surface and with appropriate washing steps, fractions of the sample can be selectively removing leaving specific components on the target for further analysis by MALDI TOF mass spectrometry. It is envisaged that this may be a useful technique to apply with the labels of this invention, both for analytical purposes and for separating labelled analytes from unreacted tags, particularly surfaces coated with cation or anion exchange resins.

Protein Expression Profiling and Peptide Mass Fingerprints

The second aspect of the invention provides methods of comparing the expression levels of one or more samples of analytes. In preferred embodiments that samples comprise polypeptides and the polypeptides in different samples are separated by 2-D gel electrophoresis and the separated polypeptides are identified using peptide mass fingerprinting. To compare the expression profile of two samples it is necessary to determine the identity and relative quantities of each of the component polypeptides in the two samples. The third aspect of this invention provides methods to determine both the identity and the relative quantities of each of the component polypeptides in two or more different samples. To achieve this the polypeptides in each sample are labelled with labels that can be resolved by their fluorescence emissions. The labelled polypeptides are then pooled. The components of the pooled samples are resolved from each other by separating the components using electrophoretic or chromatographic procedures. The separated proteins can then be identified by peptide mass fingerprinting. The use of the compounds and labelling procedures described in this invention also allows the relative levels of each component polypeptide to be determined by fluorescence measurements prior to the mass spectrometric identification of the labelled polypeptides. In addition, the tags of this invention enhance the sensitivity of the mass spectrometric identification step.

In a preferred embodiment of the second aspect of the invention, a method is provided for the analysis two or more polypeptide containing samples, each sample containing more than one polypeptide, the method comprising the steps of:
1. Covalently reacting the polypeptides of each of the samples with a tag of the form provided by the first aspect of this invention.
2. Pooling the labelled samples.
3. Separating the pooled samples by gel electrophoresis, iso-electric focusing, liquid chromatography or other appropriate means to generate discrete fractions. These fractions may be bands or spots on a gel or liquid fractions from a chromatographic separation. Fractions from one separation may separated further using a second separation technique. Similarly further fractions may be fractionated again until the proteins are sufficiently resolved for the subsequent analysis steps.
4. Digesting the polypeptide or polypeptides in each fraction with a sequence specific cleavage reagent
5. Analysing the digests by mass spectrometry, to identify the polypeptides by peptide mass fingerprinting.

In the above preferred embodiment of the second aspect of the invention, the step of fractionating the proteins is preferably effected by performing 2-dimensional gel electrophoresis, using iso-electric focusing in the first dimension and SDS PAGE in the second dimension. Typically, the gel is visualised to identify where proteins have migrated to on the gel. Visualisation of the gel is typically performed by staining the gel to reveal protein spots. However, the tags of this invention comprise fluorophores and the usual staining step can, therefore, be omitted. The proteins in each spot are thus identified by the fluorescence of the tag compounds. The gel is therefore scanned with a laser to excite the dyes—different dyes should have either a different excitation wavelength or a different emission wavelength (or both) to allow the different dyes to be imaged independently. The Cy3 compound shown in FIG. 1 has an optimum excitation wavelength of 553 nm and maximum emission at a wavelength of 569 nm while the Cy5 compound has an optimum excitation wavelength of 645 nm and maximum emission at a wavelength of 664 nm, allowing these two dyes to be used together. With these compounds, a gel would be imaged twice using a laser to excite the different dyes thus generating two different fluorescent images of the gel corresponding to each sample. Since these dyes have been optimised for co-migration the two images should be easily registered to allow the emission intensities for corresponding proteins in each sample to be compared. This information can then be used to identify proteins that are show differential expression in the two samples to be identified. This means that the subsequent identification of proteins by mass spectrometry can be made more efficient as it becomes possible to select only those proteins showing regulation for subsequent identification by peptide mass fingerprinting. There are then two approaches to the identification step. In the first approach, the proteins are extracted from the gel. Robotic instrumentation can be used to excise the protein containing spots from the gel. The proteins are then extracted from the excised gel spot. These extracted proteins are then digested and the digest peptides from the polypeptides are analysed by mass spectrometry to determine a peptide mass fingerprint, usually by MALDI TOF mass spectrometry but electrospray mass spectrometry is also quite widely used. Proteins can also be extracted by electroblotting onto a polyvinylidene difluoride membrane after which enzymatic digestion of the proteins can take place on the membrane (Vestling M M, Fenselau C, Biochem Soc Trans 22(2):547-551, “Polyvinylidene difluoride (PVDF): an interface for gel electrophoresis and matrix-assisted laser desorption/ionisation mass spectrometry”, 1994). In the second approach the polypeptides are digested in the gel, and the digest peptides are extracted from the gel or from excised gel spots for determination of peptide mass fingerprints by mass spectrometry (Lamer S, Jungblut P R, J Chromatogr. B Biomed. Sci Appl. 752(2):311-322, “Matrix-assisted laser desorption-ionisation mass spectrometry peptide mass fingerprinting for proteome analysis: identification efficiency after on-blot or in-gel digestion with and without desalting procedures.” 2001).

Peptide Mass Fingerprinting of Labelled Polypeptides

In the second aspect of this invention, a method of analysing one or more samples of analyte molecules is provided. In this method the analyte molecules are covalently labelled with a tag compound of the first aspect of this invention. The labelled analytes are separated and then optionally cleaved. The cleavage peptides are then embedded in a MALDI matrix comprising a further dye molecule, which may be the same or different from the MALDI dye that comprises the tag compound linked to the analyte molecules. The labelled and embedded biomolecules are then analysed in a MALDI mass spectrometer. The MALDI dye used to label the analyte molecule and the dye chosen as a free matrix are both chosen to absorb light strongly in the frequency used for the MALDI process. Typically, laser Ultra-Violet (UV) frequencies of 266 nm (Nd—YAG lasers) or 337 nm (Nitrogen Lasers) are used.

Peptides and proteins are preferred biomolecules that benefit from the methods of this invention. A polypeptide or peptide or mixtures of polypeptides or peptides can be isolated for analysis by any of the conventional means such as electrophoresis, chromatography or affinity chromatography. For the purposes of mass spectrometry, it is preferred that polypeptides or proteins are not contaminated with salts or detergents, particularly metal salts. Various techniques for desalting a polypeptide or peptide mixture are known in the art such as gel filtration, dialysis or the use of hydrophobic resins. A particularly convenient and simple method for de-salting peptides involves aspiration of a small quantity of a solution of the peptide or polypeptide mixture in a pipette tip incorporating C18 packing materials. Salts and detergents can be eluted first as C18 resin typically has a higher affinity for peptides than the more polar salt contaminants. This clean-up step substantially improves the detection sensitivity of the analysis of the peptides. Pipette tips pre-packaged with appropriate resins and instructions for their use are commercially available from Millipore (Bedford, Mass., USA) under the trademark ‘Zip Tip’.

Preferred UV-absorbing dyes for use with this invention include active esters of cinnamic acid and its derivatives, active esters of nicotinic acid or active esters of hydroxybenzoic acid derivatives. Thus, in one embodiment of the second aspect of this invention, isolated peptides in a mixture are labelled with a tag comprising an active ester of a 4-hydroxy-alpha-cyano-cinnamic acid derivative. The peptides are desalted using a Zip tip and then embedded in a matrix of unmodified 4-hydroxy-alpha-cyano-cinnamic acid. Typically, a solution of the matrix is prepared in a volatile solvent such as acetonitrile containing a small amount of trifluoroacetic acid (0.1 to 0.5% by volume is sufficient). This solution is then pipetted onto a metal target to form small droplets. A small quantity of the desalted, labelled peptide solution is then dropped into the droplet of matrix solution. This solution is then left to dry so that the peptides can co-crystallise with the matrix. In other techniques the matrix solution is allowed to dry and crystallise before the peptide solution is added on top (Hutchens and Yip, Rapid Commun. Mass Spectrom. 7: 576-580, 1993). This procedure may also be repeated to produce layers of co-crystallised analyte and matrix. These and other variations of the co-crystallisation technique may be used to improve the analysis of peptides or polypeptides. Liquid matrices, as discussed above may also be used. In general the success or failure of these techniques depends on the composition of the peptide mixture and so it may be necessary to try different procedures to obtain good spectra for a particular sample. The matrix/peptide co-crystals are then analysed by laser desorption in a MALDI-TOF mass spectrometer.

Quantitation Methods

The methods and compounds of the present invention are particularly well adapted to quantitation of proteins and peptides. This may be achieved in a number of different ways, the more preferred of which are discussed in the following.

A key difference in this application over the prior art is that it provides methods of enhancing MALDI sensitivity using SMTs that allow differential quantitation of proteins from different samples in the same experiment by either (a) incorporating different fluorescent dyes that can be detected and relatively quantified in the gel or (b) incorporating different mass reporters into the labels such that polypeptides with the same sequence produce PMFs offset from each other in MALDI, but whose relative ion intensities match the relative abundance of the parent protein, or (c) incorporating different mass reporters into the labels such that quantification is made in the tandem MS mode on a MALDI TOF/TOF mass spectrometer or a tandem MS machine fitted with a MALDI source.

Accordingly, the present invention provides a further method for characterising an analyte by matrix assisted laser desorption ionisation (MALDI) mass spectrometry, which method comprises:
(a) labelling the analyte with a light-absorbing label that absorbs light at a pre-determined frequency, to form a labelled analyte;
(b) embedding the labelled analyte in a matrix formed from at least one compound that absorbs light, to form an embedded labelled analyte;
(c) desorbing the embedded labelled analyte by exposing it to light having the pre-determined frequency, to form a desorbed analyte; and
(d) detecting the desorbed analyte by mass spectrometry to characterise the analyte;
   wherein the step of detecting the desorbed analyte by mass spectrometry precedes, includes, or is followed by, a step of detecting a quantity of the analyte that is present.

In method (a) described above, the light absorbing label comprises a fluorophore moiety, and prior to detecting by mass spectrometry, the analyte is selected for detection on the basis of its fluorophore moiety. The quantitation in this embodiment is performed by measuring the fluorescence of the fluorophore moiety either on a gel or in a liquid chromatography run. In this method, it is preferred that the labels are of the same mass in order that the same analytes from different samples elute together.

In method (b) described above, the analytes are labelled with different mass reporters. These may be different SMT labels (see Example 1 below) or other labels such as isotopically labelled PST labels (see Example 2 below). The quantitation is performed by combining the samples and performing mass spectrometric analysis of the combined sample mixture. The peak height/areas of the characteristic peak pairs (or more if there are more than two samples) are measured to give relative abundances for each species. The fluorophore groups are not essential to this embodiment; it is merely necessary that the light absorbing label is present to provide the correct sensitivity for the mass spectrometric analysis to be quantitatively reliable.

In method (c) described above, the analytes are labelled with different mass reporters once again. These are preferably the TMT labels described in more detail above. Quantitation is performed by employing a tandem mass spectrometric method combined with MALDI to detect the reporter group and determining the quantity of the reporter group (see Example 3 below). The fluorophore groups are not essential to this embodiment; it is merely necessary that the light absorbing label is present to provide the correct sensitivity for the mass spectrometric analysis to be quantitatively reliable.

In method (b), the light absorbing labels of the present invention may be individually distinguishable in a mass spectrum on the basis of their mass. Thus, if a protein A is present in two samples 1 and 2, from the MALDI spectrum of the samples the ions in the spectra resulting from the same fragment, but coming from different samples, will be resolved, since they will be attached to labels having different masses. These different masses may be achieved by isotopic substitution, or by small chemical alteration of the labels. An example of the former includes substituting ¹H, ¹²C, ¹⁴N and ¹⁶O with ²H, ¹³C, ¹⁵N and ¹⁸O respectively. An example of the latter would be the inclusion of an extra inert group, such as a CH₂ group, in the molecule (compare for example SMT #13 and SMT #14 in FIG. 6).

As mentioned above, an alternative method involves attaching fragments to further labels that are individually distinguishable on the basis of their mass, in addition to the sensitizer labels of the present invention. Examples of such labels are disclosed in WO 98/32876 and WO 00/20870. These labels are generally termed protein sequence tags (PSTs) and when adapted for quantitation, are termed qPSTs. In this embodiment of the present invention, if a protein A is present in two samples 1 and 2, from the MALDI spectrum of the samples the ions in the spectra resulting from the same fragment, but coming from different samples, will be resolved, since they will be attached to qSMT labels having different masses. The labels of the present invention will make quantitation more accurate by increasing sensitivity.

Method (c) involves attaching fragments to still further labels that are individually distinguishable on the basis of their mass, in addition to the sensitizer labels of the present invention. Examples of such labels are disclosed in WO 01/68664 and WO 03/025576. These labels are generally tandem mass tags (TMTs) and when adapted for quantitation, are termed qTMTs. They have the advantage that they are formed in two parts, once being a mass marker, and one being to normalise the mass so that all TMT labels in a set have the same mass. This has the advantage that during HPLC, or another chromatographic method, the labelled fragments that have the same mass will elute in the same way, even though they have different labels. In this embodiment of the present invention, if a protein A is present in two samples 1 and 2, from the MALDI spectrum of the samples the ions in the spectra resulting from the same fragment, but coming from different samples, will be resolved, since they will be attached to qTMT labels having different masses. The labels of the present invention will make quantitation more accurate by increasing sensitivity. In fact, the present labels may be used in place of TMT labels in some instances, when appropriately designed.

Mass Spectrometers

The essential features of a mass spectrometer are as follows
Inlet System->Ion Source->Mass Analyser->Ion Detector->Data Capture System

There are various inlet systems, ion sources and mass analysers that can be used for the purposes of analysing large biomolecules but in the context of this invention the ion source is a Matrix Assisted Laser Desorption ion source for which there are only a limited number of inlet systems. A variety of mass analysers, ion detectors and data capture systems may be used with MALDI although some mass spectrometer geometries are not commercially produced. Time-of-flight mass analysers are typically used with MALDI as well as Fourier Transform Ion Cyclotron Resonance mass analysers and Quadrupole/Time-of-flight mass analysers. In principle ion traps and sector instruments can be used with MALDI but generally these are not commercially produced.

Matrix Assisted Laser Desorption Ionisation (MALDI)

MALDI requires that the biomolecule solution be embedded in a large molar excess of a photo-excitable ‘matrix’. The application of laser light of the appropriate frequency results in the excitation of the matrix which in turn leads to rapid evaporation of the matrix along with its entrapped biomolecule. Proton transfer from the acidic matrix to the biomolecule gives rise to protonated forms of the biomolecule which can be detected by positive ion mass spectrometry, particularly by Time-Of-Flight (TOF) mass spectrometry. Negative ion mass spectrometry is also possible by MALDI TOF. This technique imparts a significant quantity of translational energy to ions, but tends not to induce excessive fragmentation despite this. The laser energy and the timing of the application of the potential difference used to accelerate the ions from the source can be used to control fragmentation with this technique. This technique is highly favoured for the determination of peptide mass fingerprints due to its large mass range, due to the prevalence of singly charged ions in its spectra and due to the ability to analyse multiple peptides simultaneously.

The photo-excitable matrix comprises a ‘dye’, i.e. a compound that strongly absorbs light of a particular frequency, and which preferably does not radiate that energy by fluorescence or phosphorescence but rather dissipates the energy thermally, i.e. through vibrational modes. It is the vibration of the matrix caused by laser excitation that results in rapid sublimation of the dye, which simultaneously takes the embedded analyte into the gas phase.

Mass Analysers

Time-of-Flight Mass Analysers

As the name implies, Time-of-flight mass analysers measure the time it takes for ions to travel a predetermined distance under the influence of a predetermined potential difference. The time-of-flight measurement allows the mass-to-charge ratio of ions striking a detector to be calculated. These instruments measure the arrival of almost all of the ions in a sample and as a result can be quite sensitive although, selectivity with this technique is more difficult to achieve. This technique can also detect ions with higher mass-to-charge ratios than can typically be measured in an ion trap or quadrupole mass spectrometer. TOF mass analysers are presently widely used with MALDI.

Ion Traps

Ion Trap mass analysers are related to the quadrupole mass analysers. The ion trap generally has a 3-electrode construction—a cylindrical electrode with ‘cap’ electrodes at each end forming a cavity. A sinusoidal radio frequency potential is applied to the cylindrical electrode while the cap electrodes are biased with DC or AC potentials. Ions injected into the cavity are constrained to a stable circular trajectory by the oscillating electric field of the cylindrical electrode. However, for a given amplitude of the oscillating potential, certain ions will have an unstable trajectory and will be ejected from the trap. A sample of ions injected into the trap can be sequentially ejected from the trap according to their mass/charge ratio by altering the oscillating radio frequency potential. The ejected ions can then be detected allowing a mass spectrum to be produced.

Ion traps are generally operated with a small quantity of a ‘bath gas’, such as helium, present in the ion trap cavity. This increases both the resolution and the sensitivity of the device as the ions entering the trap are essentially cooled to the ambient temperature of the bath gas through collision with the bath gas. Collisions both increase ionisation when a sample is introduced into the trap and dampen the amplitude and velocity of ion trajectories keeping them nearer the centre of the trap. This means that when the oscillating potential is changed, ions whose trajectories become unstable gain energy more rapidly, relative to the damped circulating ions and exit the trap in a tighter bunch giving a narrower larger peaks.

Ion traps can mimic tandem mass spectrometer geometries, in fact they can mimic multiple mass spectrometer geometries allowing complex analyses of trapped ions. A single mass species from a sample can be retained in a trap, i.e. all other species can be ejected and then the retained species can be carefully excited by super-imposing a second oscillating frequency on the first. The excited ions will then collide with the bath gas and will fragment if sufficiently excited. The fragments can then be analysed further. It is possible to retain a fragment ion for further analysis by ejecting other ions and then exciting the fragment ion to fragment. This process can be repeated for as long as sufficient sample exists to permit further analysis. It should be noted that these instruments generally retain a high proportion of fragment ions after induced fragmentation. These instruments and FTICR mass spectrometers (discussed below) represent a form of temporally resolved tandem mass spectrometry rather than spatially resolved tandem mass spectrometry which is found in linear mass spectrometers.

Fourier Transform Ion Cyclotron Resonance Mass Spectrometry (FTICR MS)

FTICR mass spectrometry has similar features to ion traps in that a sample of ions is retained within a cavity but in FTICR MS the ions are trapped in a high vacuum chamber by crossed electric and magnetic fields. A pair of plate electrodes that form two sides of a box generates the electric field. The box is contained in the field of a superconducting magnet which in conjunction with the two plates, the trapping plates, constrain injected ions to a circular trajectory between the trapping plates, perpendicular to the applied magnetic field. The ions are excited to larger orbits by applying a radio-frequency pulse to two ‘transmitter plates’, which form two further opposing sides of the box. The cycloidal motion of the ions generates corresponding electric fields in the remaining two opposing sides of the box, which comprise the ‘receiver plates’. The excitation pulses excite ions to larger orbits which decay as the coherent motions of the ions is lost through collisions. The corresponding signals detected by the receiver plates are converted to a mass spectrum by Fourier Transform (FT) analysis.

For induced fragmentation experiments these instruments can perform in a similar manner to an ion trap—all ions except a single species of interest can be ejected from the trap. A collision gas can be introduced into the trap and fragmentation can be induced. The fragment ions can be subsequently analysed.

### EXAMPLES

1. Quantitation Using the Tags of the Present Invention (qSMT)

In this experiment, the labels of the present invention (sensitiser mass tags, SMTs) were adapted for quantitation by using different linker chain lengths. Two labels (SMT #13 and SMT #14) were employed, as shown in FIG. 6.

A BSA digest was performed according to the method of the present invention. Two samples were employed and the lysine side-chains and N-termini in each sample were modified with one of the tags (a different tag for each sample).

A MALDI spectrum of each sample is shown in FIG. 7, and an expanded spectrum is shown in FIG. 8. It can be seen from these spectra that the same peak profile is produced, one being displaced slightly in mass to the other due to the differing mass of the two tags. This is more easily seen in the 1:1 mixture spectrum shown in FIG. 9, and the corresponding expanded spectrum shown in FIG. 10 as well as the spectra for separate samples and a 1:1 mixture shown in FIG. 11.

The quantities of the species present are accurately reflected in these spectra, as can be seen from the similar peak heights (and areas) in the above figures. The quantities of the two samples present were varied to demonstrate this. FIG. 12 shows a corresponding set of spectra to those of FIG. 11, but with a variance in quantities for three mixtures, 1:2, 1:1 and 2:1. FIG. 14 shows the same spectra after de-isotoping. The variance with quantity is quite pronounced, and can be employed to calculate absolute quantities, or to compare relative quantities between the two samples.

Measurement of the areas under the peaks in the spectra for the three quantity ratios 1:2, 1:1 and 2:1 discussed above leads to the intensity of the peaks and provided the following results:

| | | | | |
|---|---|---|---|---|
| Mass #14 | Mass #13 | Area #14 | Area #13 | IntRatio |

1:1 Ratio

| | | | | |
|---|---|---|---|---|
| 2294.5487 | 2322.5789 | 151663.2656 | 218166.8906 | 1.44 |
| 1895.1418 | 1909.1532 | 60044.4141 | 70008.8359 | 1.17 |
| 1807.1798 | 1821.1940 | 89257.2344 | 130462.4297 | 1.46 |
| 1366.8499 | 1394.8811 | 12136.5459 | 25747.8438 | 2.12 |
| 1254.7840 | 1268.8064 | 76826.4688 | 85698.9766 | 1.12 |
| 1610.8603 | 1624.8977 | 4278.52 | 5764.2 | 1.35 |
| 1628.7812 | 1656.8080 | 3729.18 | 7749.09 | 2.08 |

1:2 Ratio

| | | | | |
|---|---|---|---|---|
| 2294.4765 | 2322.5124 | 202946.4063 | 157382.4844 | 0.78 |
| 1895.0811 | 1909.1007 | 88559.1641 | 47194.8633 | 0.53 |
| 1807.1282 | 1821.1480 | 130475.9609 | 95590.7344 | 0.73 |
| 1767.1523 | 1781.1639 | 4977.0107 | 4069.8218 | 0.82 |
| 1628.9354 | 1656.9702 | 5005.8755 | 5580.9058 | 1.11 |
| 1366.8143 | 1394.8550 | 16875.4199 | 18965.0781 | 1.12 |
| 1254.7639 | 1268.7858 | 81581.2109 | 60097.9844 | 0.74 |
| 1610.8507 | 1624.8916 | 6388.12 | 3745.56 | 0.59 |
| 1628.7839 | 1656.8137 | 5797.05 | 5255.69 | 0.91 |

2:1 Ratio

| | | | | |
|---|---|---|---|---|
| 2294.5188 | 2322.5505 | 103125.0156 | 206687.4531 | 2.00 |
| 1895.1232 | 1909.1334 | 23785.6211 | 51043.1523 | 2.15 |
| 1807.1550 | 1821.1750 | 51042.6289 | 118676.7578 | 2.33 |
| 1611.0391 | 1625.0670 | 4508.1431 | 8871.6230 | 1.97 |
| 1366.8355 | 1394.8690 | 11495.3545 | 51323.0781 | 4.46 |
| 1254.7722 | 1268.7957 | 80310.8125 | 111664.5859 | 1.39 |

The IntRatio (internal ratio) is calculated as follows:
IntRatio=I_{S}/I_{L}
I_{L}=Ion intensity of the light species
I_{S}=Ion intensity of the heavy species

It is also worth noting the following:
N_{L}=amount of light species
N_{S}=amount of heavy species
Delta=I_{S}N_{L}/I_{L}N_{S}
From these small scale experiments and from the above ratios, it can be seen that a reasonable estimate of the relative quantity of the two species can be determined.
2. Quantitation Using Tags of the Present Invention in Conjunction with qPST (SMT-qPST)

In this experiment, the labels of the present invention (sensitiser mass tags, SMTs) were adapted for quantitation by using them in conjunction with qPST tags, (these tags are discussed in more detail above). A pair of qPST labels were isotopically labelled (+85 and +95 Da in mass respectively). The qPST tags were applied to separate samples prior to performing a tryptic digest. This modifies lysine residues, which are not cut by trypsin.

After the digest, the sensitiser labels were added, and these reacted with the new peptide N-termini. In each mixture, each peptide has a maximum of one SMT.

An HPLC separation was carried out on each sample, prior to performing a MALDI mass spectrometry on the samples. A spectrum of the combined samples is shown in FIG. 15, clearly highlighting the expected pairs of peaks.

The experiment was repeated with and without the sensitiser label, to demonstrate the utility of the present SMT labels. The comparative spectra are shown in FIG. 16. In the Figure, the upper spectrum comprises the sensitiser labels, whilst the lower does not. For example, the 902/907 pair in the lower spectrum was modified with sensitiser (+341 Da) and appears as the 1243/1248 pair in the upper spectrum. The peaks in the upper spectrum are significantly enhanced by virtue of the present SMT labels.

For completeness, comparison spectra after HPLC are shown in FIG. 17. FIG. 18 shows a smear at the end of the HPLC run. The pair 2066/2071 has a long elution, but this has not affected the ability of the method to provide reliable quantitation.

3. Quantitation Using Tags of the Present Invention in Conjunction with Tandem Mass Spectrometry

In this experiment, the labels of the present invention (sensitiser mass tags, SMTs) were employed using the MS/MS mode of the mass spectrometer. A peptide VATVSLPR was labelled with SMT #2 in a method of the present invention. SMT #2 has the following structure:

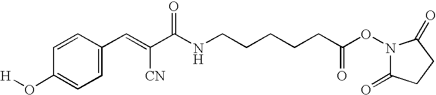

A tandem mass spectrum for the resulting fragments is shown in FIG. 19. The spectrum shows immonium ions at 70, 72, 86, 112 and 114. An ion is produced by fragmentation of the sensitiser tag at 172 (the breaking of the amide bond in SMT #2). Peaks were identified as follows:

| | |
|---|---|
| Y2-NH3 | 255.1 |
| Y2 | 272.2 |
| B1 | 384.2 |
| Y3 | 385.2 |
| B2 | 455.3 |
| B3-18 (Tyr) | 538.3 |
| B3 | 556.3 |
| B4 | 655.3 |

The nomenclature used here is standard nomenclature as devised by Roeppstorf and Fohlmann in 1984. It is used in MS/MS fragmentation of peptides, the Y referring to C-terminal fragments and the B referring to N-terminal fragments (for example, BI is the first N-terminal fragment in the spectrum).

This Example demonstrates the utility of the SMT labels of the present invention in further characterisation methods.

1. A method for characterising an analyte by matrix assisted laser desorption ionisation (MALDI) mass spectrometry, which method comprises:
(a) labelling the analyte with a light-absorbing label that absorbs light at a pre-determined frequency, to form a labelled analyte;
(b) embedding the labelled analyte in a matrix formed from at least one compound that absorbs light, to form an embedded labelled analyte;
(c) desorbing the embedded labelled analyte by exposing it to light having the pre-determined frequency, to form a desorbed analyte; and
(d) detecting the desorbed analyte by mass spectrometry to characterise the analyte;
wherein the light absorbing label comprises a fluorophore moiety, and wherein prior to detecting by mass spectrometry, the analyte is selected for detection on the basis of its fluorophore moiety.

2. A method according to claim 1, wherein the analyte is selected for detection on the basis of the identity and/or quantity of its fluorophore moiety.

3. A method according to claim 1, wherein the fluorophore moiety comprises a xanthene dye moiety, or a cyanine dye moiety.

4. A method according to claim 3, wherein the fluorophore moiety comprises a propyl-Cy3-N-hydroxysuccinimide ester, a methyl-Cy5-hydroxysuccinimide ester or a Cy2-N-hydroxysuccinimide ester.

5. A method according to claim 1, wherein the desorbed analyte is directly detected by mass spectrometry.

6. A method according to claim 1, in which the desorbed analyte is indirectly detected by mass spectrometry, wherein the analyte is additionally labelled with a mass label relatable to the analyte, and wherein the mass label is cleaved from the desorbed analyte and detected by mass spectrometry to characterise the analyte.

7. A method according to claim 1, wherein the light to which the embedded labelled analyte is exposed is laser light.

8. A method according to claim 1, wherein the compound forming the matrix absorbs light at the same frequency as the light-absorbing label.

9. A method according to claim 1, wherein the matrix and the light-absorbing label are formed from the same compound.

10. A method according to claim 1, wherein the matrix is a solid matrix or a liquid matrix.

11. A method according to claim 10, wherein the matrix is a liquid matrix comprising nitrobenzyl alcohol.

12. A method according to claim 1, wherein the matrix comprises an acid matrix or a basic matrix.

13. A method according to claim 12, wherein the matrix comprises a compound selected from 3-hydroxypicolinic acid, 2,5-dihydroxybenzoic acid and 4-hydroxy-alpha-cyanocinnamic acid.

14. A method according to claim 1, wherein the light-absorbing label is formed from a dye.

15. A method according to claim 14, wherein the dye is a non-fluorescent dye.

16. A method according to claim 14, wherein the dye comprises 4-dimethylaminoazobenzine-4′-sulphonyl chloride (DABSYL chloride), 3-hydroxypicolinic acid, 2,5-dihydroxybenzoic acid and/or 4-hydroxy-alpha-cyanocinnamic acid.

17. A method according to claim 1, wherein the analyte comprises one or more compounds selected from a protein, a polypeptide, a peptide, a peptide fragment and an amino acid.

18. A method for characterising a polypeptide, which method comprises the steps of:
(a) optionally reducing cysteine disulphide bridges in the polypeptide to form free thiols, and capping the free thiols;
(b) cleaving the polypeptide with a sequence specific cleavage reagent to form peptide fragments;
(c) optionally deactivating the cleavage reagent;
(d) capping one or more ε-amino groups that are present with a lysine reactive agent;
(e) analysing peptide fragments according to a method as defined in claim 1 to form a mass fingerprint for the polypeptide; and
(f) determining the identity of the polypeptide from the mass fingerprint.

19. A method for characterising a population of polypeptides, which method comprises the steps of:
(a) optionally reducing cysteine disulphide bridges in one or more polypeptides to form free thiols, and capping the free thiols;
(b) separating one or more polypeptides from the population;
(c) cleaving one or more polypeptides with a sequence specific cleavage reagent to form peptide fragments;
(d) optionally deactivating the cleavage reagent;
(e) capping one or more ε-amino groups that are present with a lysine reactive agent;
(f) analysing peptide fragments according to a method as defined in claim 1 to form a mass fingerprint for one or more polypeptides; and
(g) determining the identity of one or more polypeptides from the mass fingerprint.

20. A method for comparing a plurality of samples, each sample comprising one or more polypeptides, which method comprises the steps of:
(a) optionally reducing cysteine disulphide bridges and capping the free thiols in one or more polypeptides from the samples;
(b) separating one or more polypeptides from each of the samples;
(c) cleaving the polypeptides with a sequence specific cleavage reagent to form peptide fragments;
(d) optionally deactivating the cleavage reagent;
(e) capping one or more ε-amino groups that are present with a lysine reactive agent;
(f) analysing peptide fragments according to a method as defined in claim 1 to form a mass fingerprint for one or more polypeptides from the samples; and
(g) determining the identity of one or more polypeptides in the samples from one or more mass fingerprints.

21. A method according to claim 1, wherein the lysine-reactive agent is a labelled lysine-reactive agent.

22. A method according to claim 20, for comparing a plurality of samples, each sample comprising one or more polypeptides, which method comprises the steps of:
(a) optionally reducing cysteine disulphide bridges and capping the free thiols in one or more polypeptides from the samples;
(b) capping one or more ε-amino groups that are present in each sample with a labelled lysine reactive agent;
(c) pooling the samples;
(d) separating one or more polypeptides from the pooled samples;
(e) cleaving the polypeptides with a sequence specific cleavage reagent to form peptide fragments;
(f) optionally deactivating the cleavage reagent;
(g) analysing said peptide fragments to form a mass fingerprint for one or more polypeptides from the samples; and
(h) determining the identity of one or more polypeptides in the samples from one or more mass fingerprints.
wherein the same label is employed for polypeptides or peptides from the same sample, and different labels are employed for polypeptides or peptides from different samples, such that the sample from which a polypeptide or peptide originates can be determined from its label.

23. A method according to claim 18, wherein the sequence specific cleavage agent cleaves the one or more polypeptides on the C-terminal side of a lysine residue.

24. A method according to claim 18, wherein the specific cleavage reagent comprises Lys-C or Trypsin.

25. A method according to claim 18, wherein the peptide fragments having capped ε-amino groups are removed by affinity capture, and wherein the lysine reactive agent comprises biotin.

26. A method according to claim 18, wherein the lysine reactive agent comprises a hindered Michael reagent.

27. A method according to claim 26, wherein the hindered Michael agent comprises a compound having the following structure: 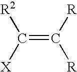
wherein X is an electron withdrawing group that is capable of stabilising a negative charge; the R groups independently comprise a hydrogen, a halogen, an alkyl, an aryl, or an aromatic group with the proviso that at least one of the R groups comprises a sterically hindering group; and the group R² comprises a hydrogen, a halogen, a hydrocarbon group, an electron withdrawing group and/or a linker capable of attachment to an affinity capture functionality or a solid phase support.

28. A method according to claim 1, wherein the step of detecting the desorbed analyte by mass spectrometry precedes, includes, or is followed by, a step of detecting a quantity of the analyte that is present.

29. A method according to claim 28, wherein the quantity of the analyte that is present is determined by measurement of fluorescence of the fluorophore group attached to the analyte.

30. A method for characterising an analyte by matrix assisted laser desorption ionisation (MALDI) mass spectrometry, which method comprises:
(a) labelling the analyte with a light-absorbing label that absorbs light at a pre-determined frequency, to form a labelled analyte;
(b) embedding the labelled analyte in a matrix formed from at least one compound that absorbs light, to form an embedded labelled analyte;
(c) desorbing the embedded labelled analyte by exposing it to light having the pre-determined frequency, to form a desorbed analyte; and
(d) detecting the desorbed analyte by mass spectrometry to characterise the analyte;
wherein the step of detecting the desorbed analyte by mass spectrometry precedes, includes, or is followed by, a step of detecting a quantity of the analyte that is present.

31. A method according to claim 30, wherein the quantity of the analyte that is present is determined by analysis of the mass spectrum.

32. A labelled analyte compound, which compound has either of the following structures:
F-D-L-A
D-F-L-A
wherein F comprises a fluorophore, D comprises a light absorbing label, L comprises a linker and A comprises an analyte.

33. A compound for labelling an analyte, which compound has either of the following structures:
F-D-L-R
D-F-L-R
wherein F comprises a fluorophore, D comprises a light absorbing label, L comprises a linker, and R comprises a reactive functionality for attaching the compound to an analyte.

34. A compound according to claim 32, wherein D comprises a non-fluorescent dye.

35. A compound according to claim 32, wherein D comprises a cinnamic acid derivative, a nicotinic acid derivative, a picolinic acid derivative, a hydroxybenzoic acid derivative, a methoxybenzoic acid derivative or a sinapinic acid derivative.

36. A compound according to claim 34, wherein the non-fluorescent dye comprises a compound selected from 4-dimethylaminoazobenzine-4′-sulphonyl chloride (DABSYL chloride), 3-hydroxypicolinic acid, 2,5-dihydroxybenzoic acid and 4-hydroxy-alpha-cyanocinnamic acid.

37. A compound according to claim 32, which compound further comprises a mass marker M attached via a further linker, wherein M is selected from a compound formed from an aryl ether, and an oligomer formed from 2 or more aryl ether units.

38. A compound according to claim 32, wherein the linker, and/or the further linker, comprises a group selected from —CR₂—CH₂—SO₂—, —N(CR₂—CH₂—SO₂—)₂, —N H—CR₂—CH₂—SO₂—, —CO—N H—, —CO—O—, —NH—CO—NH—, —NH—CS—NH—, —CH₂—NH—, —SO₂—NH—, —NH—CH₂—CH₂— and —OP (═O)(O)O—.

39. A compound according to claim 32, wherein A is selected from a protein, a polypeptide, a peptide, a peptide fragment and an amino acid.

40. A compound according to claim 32, wherein F comprises a xanthene dye moiety, or a cyanine dye moiety.

41. A compound according to claim 40, wherein the fluorophore moiety comprises a propyl-Cy3-N-hydroxysuccinimide ester, a methyl-Cy5-hydroxysuccinimide ester or a Cy2-N-hydroxysuccinimide ester

42. A compound according claim 32, wherein R is for attaching the compound to a protein, a polypeptide, a peptide, a peptide fragment or an amino acid.

43. A compound according to claim 33, wherein R comprises an ester group, an acid anhydride group, an acid halide group such as an acid chloride, an N-hydroxysuccinamide group, a pentafluorophenyl ester group, a maleimide group, an alkenyl sulphone group, or an iodoacetamide group.

44. A compound according to claim 32, which compound further comprises an affinity ligand.

45. A compound according to claim 44, wherein the affinity ligand comprises biotin.

46. A compound according to claim 32, which compound further comprises an ionisable moiety.

47. A compound according to claim 46, wherein the ionisable moiety is selected from a tertiary amino group, a guanidino group and a sulphonic acid group.

48. A compound according to claim 32, which compound comprises a cinnamic acid functionality.

49. An array of two or more compounds for labelling an analyte, wherein the compounds in the array are compounds as defined in claim 33, and wherein each compound in the array is distinguishable from all other compounds in the array on the basis of its fluorophore and/or its mass.

50. A kit for characterising an analyte by matrix assisted laser desorption ionisation (MALDI) mass spectrometry, which kit comprises:
(a) one or more light absorbing labels having a reactive functionality for attaching the labels to an analyte, as defined in claim 33
(b) a compound for forming a matrix, which compound absorbs light at the same frequency as the light-absorbing label.

51. A kit for characterising an analyte by matrix assisted laser desorption ionisation (MALDI) mass spectrometry, which kit comprises:
(a) a compound, or an array of compounds, as defined claim 33;
(b) an ion exchange resin.

52. A kit according to claim 51, wherein the compound or array of compounds comprises an ionisable moiety that forms a positive charge, and wherein the ion exchange resin comprises a cation exchange resin.

53. A kit according to claim 51, wherein the compound or array of compounds comprises an ionisable moiety that forms a negative charge, and wherein the ion exchange resin comprises a anion exchange resin.
